(19) United States
(12) Patent Application Publication
(10) Pub. No.: US 2023/0190704 A1
(43) Pub. Date: Jun. 22, 2023

(54) DRUG DELIVERY SYSTEM BASED ON CALCIUM PHOSPHATE NANOPARTICLES FUNCTIONALIZED WITH BIOACTIVE COMPOUNDS FROM EUPHORBIA EXTRACT AND THE USES THEREOF

(71) Applicants: CELLBITEC, S.L., Almeria (ES); UNIVERSIDAD DE GRANADA, Granada (ES)

(72) Inventors: Francisco BERMUDEZ PEREZ, Almeria (ES); José Carlos PRADOS SALAZAR, Granada (ES); Consolacion MELGUIZO ALONSO, Granada (ES); Jusus M PORRES FOULQUIE, Granada (ES); Cristina MESAS HERNANDEZ, Granada (ES); Rosario MARTINEZ MARTINEZ, Almeria (ES); Milagros GALISTEO MOYA, Granada (ES); Raul ORTIZ QUESADA, Granada (ES); Laura CABEZA MONTILLA, Granada (ES); Maria LOPEZ-JURADO ROMERO DE LA C, Granada (ES); Antonio Femando MURILLO CANCHO, Almeria (ES); Jose Manuel DELGADO LOPEZ, Granada (ES); Victor GARCES ROBLES, Granada (ES); Gloria Belen RAMIREZ RODRIGUEZ, Granada (ES)

(21) Appl. No.: 17/926,120

(22) PCT Filed: May 5, 2021
(86) PCT No.: PCT/EP2021/061828

§ 371 Date: Nov. 17, 2022

(30) Foreign Application Priority Data
May 18, 2020 (ES) E202030454

(51) Int. Cl. A61K 31/37; A61K 36/47; A61K 9/51; A61K 31/7048; A61K 31/7034; A61P 35/00

(52) U.S. Cl. CPC A61K 31/37; A61K 9/5115; A61K 31/7034; A61K 31/7048; A61K 36/47; A61P 35/00; A61K 2236/15; A61K 2236/53; A61K 2236/333

(57) ABSTRACT

The present invention relates to a composition comprising the bioactive molecules esculetin and euphorbetin, which present antitumor activity, so it may be used for the treatment and/or prevention of cancer, especially for colorectal, pancreatic and glioblastoma. Furthermore, it relates to a drug delivery system composed of nanoparticles of calcium phosphate functionalized with said bioactive molecules, preferably obtained from a Euphorbia extract. The invention also refers to the obtaining of an ethanolic extract of plant origin from defatted flour of mature seed of Euphorbia.

### FIELD OF THE INVENTION

The present invention relates to a composition containing esculetin and euphorbetin, bioactive compounds from Euphorbia plant, a drug delivery system composed of nanoparticles of calcium phosphate functionalized with bioactive compounds from a Euphorbia extract. Both the composition and the drug delivery system present demonstrated antitumoral properties. The invention also refers to the obtaining of an ethanolic extract of plant origin to be used as an antitumoral agent, from defatted flour of mature seeds of Euphorbia. Therefore, the present invention may be included in the field of pharmacology and pharmaceutical sciences.

### BACKGROUND OF THE INVENTION

Cancer is understood as a group of diseases characterized by various alterations at the cellular level that lead to an excess proliferation and survival of malignant cells, causing abnormalities in the functioning of the organism. Among the different types of tumors, we can highlight colorectal cancer (CRC), a special object of study in this application, due to its incidence and the lack of adequate treatment, especially in the more advanced stages of the disease. The incidence of CRC worldwide is so high that it is considered a real health problem. As the third most common cancer and the fourth leading cause of cancer-related death in the world, it accounts for between one and two million new cases each year. Its incidence has increased by more than 200,000 new cases per year between 1990 and 2012, being detected more frequently in Western countries. In Spain, the diagnosed cases of cancer in general in 2017 have been 228,482 being the progression of this disease is unstoppable as shown by the forecast made for the year 2035 that places the number of new cases at 315,413. Among them, CRC has the highest incidence (15% of the total - 34 331 cases detected in 2017), followed by prostate, lung, breast, bladder and stomach. According to sex, this type of tumor is the second most frequently diagnosed in men after prostate cancer and in women it also ranks second after breast cancer. In terms of mortality, colorectal cancer is in second position in both men and women with 15,923 deaths per year, followed by pancreatic cancer. In relation to risk factors, the main factor influencing this type of tumor pathology is age, being diagnosed in people over 50 years of age (90% of cases) without other pathologies, clinics or predisposing diseases. However, persons with a family history of CRC, intestinal polyps or inflammatory bowel disease should be considered at high risk. It should be emphasized that excessive alcohol consumption, overweight and obesity, smoking, physical inactivity and certain types of food such as processed meat have been linked to this pathology.

The therapy of CRC, despite the most recent advances, has not achieved significant results, especially in the most advanced phase of the disease in which metastatic expansion of the tumor occurs, which has a decisive influence on patient survival. This expansion occurs preferentially towards the liver, the only possible treatment being 5-fluorouracil (5FU) associated or not with surgery and other agents such as irinotecan, capecitabine or oxaliplatin, or more recently, monoclonal antibodies such as cetuximab and bevacizumab (Labianca, 2010) or regorafenib and TAS-102 (Loree et al., 2017). Despite this, and as clearly indicated by the median survival of these patients (15 to 20.5 months), the results are very limited (Berrino et al., 2007). The improvement of their prognosis therefore requires the development of new strategies that add therapeutic activity to preventive action (Franceschi et al., 2012).

The development of new therapeutic strategies encompasses a large number of research fields ranging from nanotechnology to the use of systems that activate the immune system or the use of extracts from different origins, among others, which can help to improve the response to treatment. In this context, currently the activity of plant extracts or derivatives on the viability and survival of tumor cells is gaining great interest (Goyal et al., 2017), although as early as 1960 the National Cancer Institute (USA) began to evaluate the antitumor and preventive activity of different plant extracts against different carcinomas (Huang et al, 2013). Plants in general and their extracts in particular have great applications in medicine such as: i) being a direct source of therapeutic agents, ii) raw material for the manufacture of more complex semisynthetic drugs, iii) providing a chemical structure of their active principles that can serve as a model for the development of synthetic drugs iv) using these principles as taxonomic markers in the search for new drugs. These potential applications are due to the phytochemicals present in plants and their extracts. More than 5000 phytochemicals have been identified in seeds, fruits, roots, tubers, leaves, etc, which are classified as phenolic compounds, carotenoids, vitamins, alkaloids, nitrogenous compounds and organosulfur compounds (Thapliyal et al., 2018).

Phenolic compounds have attracted the interest of the scientific community due to their great structural diversity, as well as their broad bioactivity. Phenolic compounds have essential functions in plant reproduction and growth, act as defense mechanisms against pathogens, parasites and predators, and are responsible for providing plant color. They are not only beneficial to plants, but also play an important role in human health, as antioxidants, anticarcinogenic, antibacterial and anti-inflammatory (Huang et al., 2013).

Due to their bioactivity, there are numerous drugs and patents whose main components are polyphenols. This is the case of Neumentix, a patented supplement obtained by KeminTM (represented in Spain by Univar), in which it performs a drying process of harvested spearmint (Kl110 and Kl42 ranges) prior to the extraction of polyphenols, among which rosmarinic, salvianolic and caftaric acids stand out. This polyphenol-rich supplement demonstrates, through clinical studies, benefits in cognitive performance. It also shows that the polyphenols that characterize this supplement act as antioxidant agents reducing oxidative stress, promoting neuronal growth and protecting nerve cells in the brain. Similarly, polyphenols found in grapes, blueberries and other fruits and vegetables have been studied for their ability to decrease the risk of developing neurodegenerative diseases. In addition, they have been used as a supplement to minimize the effects of aging, especially memory loss, as in the case of Curcumin Optimized with NeurophenolTM (a proprietary blend of blueberry and grape extracts), from Douglas Laboratories® (Valencia, Spain: https://www.douglaslabs. en/) Patented polyphenolic extracts from grape seed (Vitaflavan®, a product of DRT- Les Derives Resiniques et Terpeniques, Dax, France: https://www.vitaflavan.com/es/), red grape pomace (Emitol®) and red wine (Provinols®, from Sucren/Vitimed, distributed by Seppic, La Garenne Colombes, France: https://www.seppic.com/provinolstm-0) are also marketed with antioxidant properties.

In the last 20 years, more than 25% of medicines are derived from plants, while another 25% are derived from modified natural products (Amin et al., 2009). It is worth mentioning that only 5% to 15% of medicinal plants have been investigated for their bioactive compounds. This highlights the importance of the search for new drugs from plant species (Rivas-Morales et al. 2016; Wong et al.,2018).

The genus Euphorbia (Euphorbiaceae), the largest of flowering plants, has been of great interest since antiquity as evidenced by the descriptions of E. helioscopia L. (Levey, 1966) and euphorbia (gum of E. resinifera Berg.) by Hippocrates, Galen and Dioscorides (Hargreaves, 1968; Stannard, 1964) and Carl Linnaeus’ description of the genus Euphorbia (Nambudiri and Nambudiri, 2013). Today we know that Euphorbia esula L. possesses a diterpene with proinflammatory and antitumor biological activity (Evans and Taylor, 1983; Kupchan et al., 1976) and that ingenol mebutate (Picato®, distributed by Laboratorios Leo Pharma, Barcelona, Spain), a diterpene isolated from Euphorbia peplus L., is used for the topical treatment of actinic keratosis and skin cancer (Berman, 2012). However, most of the studies conducted have focused on the presence of biologically active natural products in latex (Shi et al., 2008; Vasas and Hohmann, 2014), compounds that, being a rich source of diterpenes and triterpenes, have been related to not only antitumor and anti-inflammatory properties but also contraceptive and fibrinolytic properties.

Euphol, a tetracyclic triterpene alcohol present in Euphorbia sap, has been shown to be the predominant compound in the latex of all Euphorbia species (Cruz L et al., 2018) and possesses the properties described above (anti-inflammatory, contraceptive, etc). In fact, Silva et al. (2018) have recently demonstrated how this compound possesses antitumor activity in 15 in vitro models with tumor cells in which the inhibitory dose 50 (IC50) was between 2-30 µM. Although it is true that the most numerous studies have been carried out with the latex, some studies have analyzed other areas of the plant. Thus, the seeds of Euphorbia lathyris, one of the best-known species of this genus, have been traditionally used in medicine to treat dropsy, ascites, constipation, amenorrhea and scabies from ethanolic extracts of dried seed (Liao S et al., 2005). Euphorbia seeds contain a large amount of natural diterpenoids, among which the so-called Euphorbia factors L1-L28. Bicchi et al. (Bicchi et al., 2001), for example, have described the presence of diterpenoid fractions corresponding to ingenol and Euphorbia factors in the seed oil of Euphorbia lathyris. In the same line, the paper by Jiao W; et al. (2010) analyzes an ethanolic extract of Euphorbia lathyris seeds identifying 22 types of compounds, such as diterpenoids, triterpenoids, steroids, fatty acid esters, coumarins as esculetin. In particular, the following are cited: 20-O-hexadecanoyl-ingenol (4), 3-O-hexadecanoyl-ingenol (5), 15,17-O-diacetyl-3-O-cinnamoyl-17-hydroxyquinol (6), 5,15,17-O-triacetyl-3-O-benzoyl-17-hydroxyisolatirol (7), 5,15-O-diacetyl-3-O-nicotinoyl-latirol (8), 5,15-O-diacetyl-3-O-benzoyl-7-O-nicotinoyl-7-hydroxy-latirol (9), ingenol (10), latirol (11), esculetin (12), β-sitosterol (13), benzene-1,2,3-triol (14), palmitic acid (15), 2,3-dihydroxypropyl icosanoate (16), 2,3-dihydroxypropyl oleate (17), 2,3,4-trihydroxybutyl hexadec-3-enoate (18), aurantianide acetate (19), benzoic acid (20), p-hydroxybenzoic acid (21), oleic acid (22).

Compounds extracted from the seeds have shown high cytotoxicity against cancer cells. Thus, for example, Teng et al. (Teng Yu-Ning et al., 2018), similarly to Meng et al. (Meng et al., 2013), describe the preparation of an ethanolic extract of Euphorbia lathyris seeds, by a process using 95% ethanol at reflux, for several hours; they also describe a petroleum ether partition. The main compounds present in the extract obtained by these authors are lathyran-type diterpenoids, with five diterpenes with lathyran structure (the Euphorbia factors L1, L2, L3, L8 and L9) showing cytotoxic activity against lung carcinoma, nasopharyngeal carcinoma, breast cancer and triple-negative breast cancer cell lines. Duan et al. (Duan et al, 2014), meanwhile, state in their introduction that Euphorbia seeds are already known to have a significant effect in the treatment of leukemia, carcinoma and skin cancer; they describe in the same article the preparation of an extract from Euphorbia lathyris seeds and analyze the components of a petroleum ether extract from these seeds, identifying in the same ten compounds derived from lathyran.

Fan et al. (2019), in a recent study conducted only on L2 factor isolated from Euphorbia seeds, found that it possesses antiproliferative activity against a very specific type of tumor such as hepatocellular carcinoma. Multidrug resistance (MDR) and P-glycoprotein modulating effects have also been demonstrated for Euphorbia lathyris extracts (Wang Q et al., 2018).

Therefore, Euphorbia seed, from which many biochemical components have been identified and isolated, possesses extensive pharmacological activity, but new chemical compounds as well as the exact pharmacological and toxicological mechanisms still need to be explored (Zhu et al., 2018). Authors such as Zhang et al, 2017, besides referring to previously known phytochemical components of Euphorbia lathyris such as diterpenes, euphorbetin, esculetin, daphnetin, beta-sitosterol, kaempferol-3-gucluronide, vitexcarpin, artementin, daucosgterol, p-hydroxybenzoido, flavones or flavonol glycosides, already describe different components of the phenolic acid and flavonoid type, such as gallic acid, chlorogenic acid, vanillic acid, ferulic acid, p-coumaric acid or caffeic acid, in the root, stem and seed of Euphorbia lathyris, several of which have antioxidant capacity, although they use an extraction method based on acetone, which is highly toxic for the cells, in addition to including an extraction with ethyl acetate and they do not carry out studies of antitumor capacity. Other authors, such as Nam and Lee (2000), tested the antitumor capacity of Euphorbia lathyris extracts and found some antiproliferative activity. However, the extracts are made with methanol, which is highly toxic and therefore of little use for in vivo application. In addition, the activity is demonstrated only in lung cancer cells in vitro, not detecting antitumor activity for colon cancer. On the other hand, Sdayria et al, 2018 show, through in vitro and in vivo studies, the antioxidant, antinociceptive and anti-inflammatory effects presented by polyphenolic compounds (gallic acid, epicatechin, coumaric acid, apigenin, naringenin, rutin, quercetin and kaempferol) present in the extracts obtained by processing with methanol (methanolic extracts) from the leaves of Euphorbia retusa, considering this methanolic extract an effective agent for the treatment of pain and inflammation since it inhibits some inflammatory mediators such as MDA and COX-2 and increases the activity of SODm CAT and GPx in the liver.

Components present in Euphorbia lathyris and that could be related to an antiproliferative activity, such as the polyphenols esculetin, euphorbetin, gaultherin, nicotiflorin (Kaempferol-3-ruthinoside) and carnosol have been analyzed by some authors (Lee et al., 2017 and 2019; Turkekul et al., 2018; Aliebrahimi et al., 2018; HefnyGad et al., 2018) independently. The antitumor activity studies reflected in those papers were performed with the compounds purchased from commercial houses, not with extracts of Euphorbia lathyris. Thus, for example, Lee et al. (2017 and 2019) tested Esculetin, a coumarin derivative, in cells derived from non-small cell lung cancer, evidencing an antitumor effect against such cancer. Similarly, Wang et al. (2002) tested this molecule against leukemia cells (HL-60) and observed an increase in apoptosis. However, none of these authors use Euphorbia lathyris extracts in their studies, which focus on the exposure of tumor cells to the described molecule acquired commercially and therefore without interaction with other molecules.

Thus, it can be said that in the existing bibliography so far, extracts made using highly toxic organic solvents and/or extraction processes of great complexity and duration appear after which extracts are obtained with a great variety of components (flavonoids, coumarins and terpenoids) that can increase the toxicity of these extracts. The anticancer activity of some of these extracts has been tested against cell lines of several specific types of cancer, such as non-small cell lung cancer, nasopharyngeal carcinoma, breast cancer and triple negative breast cancer, and it is also known that several of the compounds included in these extracts have anticancer properties against different types of cancer. However, the possible activity of Euphorbia lathyris seed extracts in vivo has not been tested, possibly due to the risk of toxicity, nor has it been proven whether any of these extracts could have activity against cancers as common as colon or pancreatic cancer, or as aggressive and difficult to control as glioblastoma. It would be interesting to be able to develop a process of extraction of Euphoriba lathyris seeds using non-toxic solvents and which would also give rise to extracts with a more specific composition of active substances and low toxicity such as the polyphenols of this plant, especially because this process, besides being simple to carry out, would have the advantage that the use of the extract obtained could be considered for the development of drugs with proliferative activity against the types of cancer mentioned.

The success of chemotherapy against tumors will depend on both the discovery of new anticancer agents and new systems to improve their transport. In this context, new strategies for colon cancer treatment have been investigated using nanoparticles (NPs) as drug carriers. These molecules have been shown to improve drug solubility and cell internalization, avoid multidrug resistance mechanisms (e.g. P-glycoprotein) and enabling surface functionalization for specific targeting to tumor cells. In addition, the accumulation of NPs in tumor tissues is higher than in healthy tissues reducing their toxicity. This differential behavior can be explained by the small size and physicochemical properties of NPs, as well as by the enhanced permeability and retention effect (EPR) or passive targeting. This increased EPR in tumors has been related to an inefficient lymphatic drainage and the presence of fenestrated and more permeable blood vessels. On the other hand, the activity of plant extracts or derivatives on the viability and survival of tumor cells is currently gaining great interest both to the development of new therapeutic cancer strategies and to its prevention.

With this in mind, a large variety of nanoparticles have been engineered as promising tools for cancer diagnosis and therapy in the last years. Nowadays, there are 28 commercially available drugs that already include nanoparticles in their formulations and over 45 other new nanoformulations, such as LEP-ETU®, EndoTAGR®-I and NK105, are currently in advanced phases of clinical trials. A wide variety of nanoparticles including liposomes, micelles and polymeric nanoparticles are being applied as cancer medicines, iron-replacement therapies, imaging agents, vaccines, anesthetics, fungicides or macular regenerators. Of the nine approved nanomedicines which are being currently used for cancer therapy, none incorporates biomimetic calcium phosphate nanoparticles. The first FDA-approved nano-drug, Doxil® includes doxorubicin associated with a liposome and has been widely used in ovarian and breast cancer, mieloma multiple and Kaposi sarcoma. Abraxane®, a nanoformulation that includes paclitaxel, has been widely used in breast and lung cancer, among others. VYXEOS are 100 nm bilamellar liposomes intravenously administered, which combine two chemotherapies for the treatment of acute myeloid leukemia. NBTXR3/Hensify is a 50 nm crystalline hafnium oxide nanoparticle which was approved for intratumoral administration to enhance external radiotherapy. Curiously, although there are many undergoing clinical trials involving nanoformulations, the number of trials evaluating them in CRC is more limited. Despite recent studies such as Thermodox®, a smooth-thermosensitive liposomal DOX (NCT01464593) or Promitil®, a mitomycin C nanoformulation consisting of PEGylated liposomes (NCT01705002), no nanoformulation with clinical application has been developed in this type of tumor (Cabeza et al., 2020).

Two very important aspects of using nanoparticles as drug delivery systems and/or probes for bioimaging are the biocompatibility of their composition and their specific uptake by target cells. The first step must be the use of totally safe nanoparticles. The cellular uptake process involves the active internalization of the nanoparticles inside the cells in suitable quantity and form. From the multitude of reported diverse types of nanoparticles, calcium phosphates nanoparticles, the main inorganic component of mammals hard tissues (e.g., bone and teeth), are very promising due to their remarkably biocompatibility. Indeed, nanocrystalline apatite (Ap), or its precursor, amorphous calcium phosphate (ACP) have been widely used as bioactive components of bioceramics for tissue regeneration. Features of these nanoparticles as their biodegradability, lack of toxicity, low-cost synthesis and pH-dependent solubility (i.e., slightly soluble at acidic pHs) are also very attractive for the controlled delivery of active species. Moreover, the high surface reactivity of these nanoparticles favors the electrostatic or chemical interaction with a large variety of molecules. By finely tuning the experimental conditions, the surface of the nanoparticles can be specifically enriched with molecules exerting antitumor activity. In addition, the pH-dependent dissolution of the nanoparticles allows the gradual release of the adsorbed bioactive compounds at slightly acidic pHs, for instance as that usually found at the microenvironment of the tumors (Rodríguez-Ruiz et al., 2013).

As previously mentioned, Euphorbia, has a great interest for its recognized anticancer properties. Interestingly, esculetin, a natural coumarin derivative, has been found in various plants from Euphorbia genus, together or separately from its dimer, euphorbetin, which has also been identified in some species of Euphorbia. Antiproliferative and apoptotic response in several cancer cell lines treated with esculetin has been previously reported in studies aimed to develop new cancer therapies, (Arora et al, 2016). Furthermore, the therapeutic effect can be increased by a higher relative concentration of the bioactive compounds on the surface of nanoparticles as compared to the native extract. Incorporating bioactive compounds in nanoparticles could protect these molecules from degradation by cellular metabolism and thus improve their biodistribution and bioavailability.

### DESCRIPTION OF THE INVENTION

The present invention refers to a composition with a combination of the bioactive molecules esculetin and euphorbetin which presents an antiproliferative additive effect provided by each of said molecules. The origin of these bioactive compounds may be synthetic or obtained from an extract, preferably an ethanolic extract from a vegetable specie from the Euphorbia genus (Euphorbiaceae), more specifically, from ripe seed flour of Euphorbia lathyrism, following an extraction process, which is also part of the invention and is described below.

In response to the potential drawbacks presented by the extracts of this seed described in the state of the art, which are commonly obtained with toxic solvents that hindered their in vivo application, an extract has been developed which presents favorable bioactivity and can be obtained using simple extraction processes, of short duration and using non-toxic solvents, while allowing adequate extraction yields. This new extract also presents a more specific composition of active substances and low toxicity such as Euphorbia lathyris polyphenols.

The invention also refers to a drug delivery system consisting of non-toxic and biodegradable calcium phosphate nanoparticles (CP—NPs) loaded with esculetin and euphorbetin molecules, said molecules being of synthetic origin or from a natural extract, such as the ethanolic extract obtained with the process of the invention. The nano-assamblies have been fully characterized and the loading capacity of the nanocarrier calculated. The cellular uptake and the cytotoxic activity of bioactive compounds-loaded nanoparticles were analyzed in vitro against human colon carcinoma cells, as detailed in the examples of the invention.

Thus, in a first aspect, the invention refers to a composition characterized in that it comprises:
a) 0.2 to 30 mg of esculetin per g of total composition and
b) 2 to 15 mg of euphorbetin per g of total composition.

Preferably, the composition of the invention comprises from 2 to 25 mg of esculetin per g of total composition, more preferably from 10 to 20 mg of esculetin per g of total composition. The most preferred composition comprises 19.18 mg esculetin per g total composition.

Preferably, the composition of the invention comprises from 5 to 10 mg of euphorbetin per g of total composition. The most preferred composition comprises 7 mg euphorbetin per g total composition.

The esculetin and euphorbetin molecules contained in the composition of the invention may be synthesized or obtained from natural sources in a form of ethanolic extract.

Preferably, the composition of the invention is in the form of an ethanolic extract of a plant from genus Euphorbia, more preferably the plant from genus Euphorbia is selected from Euphorbia lathyris, Euphorbia angulata, Euphorbia cyparissias, Euphorbia dulcis, Euphorbia helioscopia, Euphorbia maculata, Euphorbia peplus, Euphorbia prostrata, Euphorbia valliniana or Euphorbia verrucosa, being the most preferred Euphorbia lathyris.

Preferably, the composition of the invention when it is obtained from a Euphorbia ethanolic extract, further comprises gaultherin, kaempferol-3-rutinoside and carnosol.

In another aspect, the invention relates to a process for obtaining an ethanolic plant extract from mature seeds of Euphorbia, preferably selected from the list above and more preferably of Euphorbia lathyris, comprising the steps of:
a) grinding the seed to obtain flour;
b) extracting the flour from step a) by means of a cold hydroalcoholic extraction solution at acid pH; and
c) optionally, defatting the mature seed by mechanical cold pressing prior to steps (a) and (b).

Preferably, the process of the invention is carried out under the following conditions:
a) the seed is ground to obtain flour with a particle size of between 100 µm and 150 µm; and
b) the flour obtained in step a) is extracted with the hydroalcoholic extraction solution under the following operating conditions:
   i. temperature equal to 4° C.,
   ii. under nitrogen atmosphere,
   iii. the extraction solution is composed of ethanol, double distilled water and hydrochloric acid in proportions 50:50:0.2 by volume,
   iv. pH equal to 2,
   v. the mixture of the flour and the extraction solution is kept in stirring for 30 minutes after having reached conditions i to iv
and wherein the ethanolic extract is obtained by centrifuging the mixture of the extraction solution and the flour and collecting the supernatant.

It is especially preferred that, whatever the specific conditions of the flour obtaining and extraction stages, defatting is carried out prior to obtaining the flour at a temperature of between 40 to 50° C. and with an extraction speed of 2 to 3 kg of seed/hour.

In another preferred embodiment, also combinable with any of the others, a new extraction is carried out on the residue resulting from the first extraction, specifically on the precipitate resulting from obtaining an initial extract after centrifugation, applying the following sub-steps:
vi. the precipitate resulting from centrifuging the mixture of flour and extraction solution is resuspended in extraction solution,
vii. the suspension obtained is again kept in stirring for 30 minutes under the conditions of step b) as defined previously,
viii. the suspension is subjected to centrifugation and the supernatant is collected, and
ix. ethanolic extract results from mixing the supernatant obtained in viii) with the first supernatant obtained.

The process of the invention may contain an additional final stage, also combinable with any of the possible embodiments, in which ethanol is partially or totally evaporated from the ethanolic extract obtained.

In another aspect, the invention relates to an ethanolic extract of mature seeds of Euphorbia, preferably selected from the genus mentioned above and more preferably of Euphorbia lathyris said extract being rich in polyphenols. This extract will be obtainable by the method of the present invention.

The polyphenol-rich ethanolic extract may have a total polyphenol content ranging from 15.64 to 39.31 µg gallic acid equivalents/mg extract and/or a reducing capacity ranging from 9.43 to 24.87 µg gallic acid equivalents/mg extract. In possible embodiments, the total polyphenol content may be 15.85 ± 0.21 µg gallic acid equivalents/mg extract or 33.52 ± 5.79 µg gallic acid equivalents/mg extract, and the reducing capacity may be 9.71 ± 0.28 µg gallic acid equivalents/mg extract or 22.95 ± 1.92 µg gallic acid equivalents/mg extract.

In another possible embodiment, compatible with the above, the ethanolic extract comprises at least one polyphenol selected from the group of esculetin, euphorbetin, gaultherin, nicotiflorin (kaempferol-3-rutoside) and carnosol, preferably at least two polyphenols selected from the group of esculetin, euphorbetin and kaempferol-3-rutoside (nicotiflorin), being possible combinations for the definition of the extract the presence in the same of esculetin and euphorbetin, euphorbetin and kaempferol-3-rutoside (nicotiflorin), esculetin and kaempferol-3-rutoside (nicotiflorin) and, particularly, the presence of these three polyphenols, esculetin, euphorbetin and kaempferol-3-rutoside (nicotiflorin), especially if they appear as majority polyphenols and more especially, if it is defined that the extract comprises also at least one of the other polyphenols selected from the group of gaultherin and carnosol or more, preferably, both. Thus, it is an especially preferred embodiment of the extract of the present invention, that the same comprises the polyphenols esculetin, euphorbetin, gaultherin, kaempferol-3-ruthinoside (nicotiflorin) and carnosol. In any of these definitions, it is preferred that esculetin be present and at a concentration of (i) the ranges of 985.9 µg/L to 1197.2 µg/L and 2041.5 µg/L to 2325.8 µg/L weight of compound : volume of extract, all values inclusive, and/or (ii) 0.4 ± 0.05 mg of compound per 100 mg of extract or 0.21 ± 0.023 mg of compound per 100 mg of extract, and/or that kaempferol-3-ruthinoside (nicotiflorin) is present and a concentration of: (i) the ranges of 59.8 µg/L to 61.1 µg/L and 188.3 µg/L to 354 µg/L, weight of compound : volume of ethanolic extract, all values inclusive, and/or (ii) 0.02 ± 0.003 mg of compound per 100 mg of ethanolic extract or 0.21 ± 0.023 mg of compound per 100 mg of ethanolic extract.

As in the case of the process of the invention, and in the other aspects of the invention described below, embodiments corresponding to defatted mature seeds are preferred. Thus, in the case of the ethanolic extract of the invention, it is preferred that it complies the definition in terms of the biochemical parameters obtainable for defatted mature seeds, i.e., the extract wherein:
(i) the total polyphenol content is 3.52 ± 5.79 gallic acid equivalents/mg extract and/or the reducing capacity is 22.95 ± 1.92 µg gallic acid equivalents/mg extract,
(ii) the extract comprises the polyphenols esculetin, euphorbetin, gaultherin, kaempferol-3-ruthinoside (nicotiflorin) and carnosol,
(iii) esculetin is present at a concentration between 2041.5 µg/L and 2325.8 µg/L (w/V - weight of compound:volume of extract), both values included, and/or at a concentration of 0.21 ± 0.023 mg compound per 100 mg extract, and
(iv) kaempferol-3-rutoside (nicotiflorin) is present at a concentration of between 188.3 µg/L to 354 µg/L (w/V - weight of compound:volume of extract), both values included, and/or at a concentration of 0.02 ± 0.003 mg of compound per 100 mg of extract.

In any of the definitions, it is preferred that the extract obtained by the process of the present invention, in its most general definition or in any of its possible embodiments; included therein is the possibility of the optional final additional stage being carried out in which the final evaporation, partial or total, of the ethanol is carried out. It is preferred, again, that the stage of defatting the mature seed by mechanical cold pressing has been carried out before carrying out stage a) of milling the seed and stage b) of extracting the flour obtained and, especially, that each of the possible stages (defatting, milling and extraction proper) are carried out with the defining characteristics expressed in describing the possible realizations of the method of the invention, including the carrying out of a second extraction on the precipitate resulting from the centrifugation giving rise to the initial extract.

Another aspect of the invention refers to a drug delivery system comprising a plurality of calcium phosphate nanoparticles (CP—NPs) surface functionalized with the composition described above or with the ethanolic extract of Euphorbia described above, characterized in that said CP—NPs comprise esculetin and euphorbetin adsorbed to the surface of the nanoparticle.

In the present invention, the term “drug delivery system” refers to a formulation that enables a therapeutic substance to selectively reach its site of action without reaching the nontarget cells, organs, or tissues. Applied to the present invention, the drug delivery system is formed by the CP—NPs which act as carriers of the bioactive molecules esculetin and euphorbetin, said molecules being progressively released from the surface of the CP—NPs to the physiological target, such as tumoral cells.

The core CP—NPs may be obtained following a precipitation method known in the art, starting from solutions containing precursor salts such as K₂HPO₄, Na₂CO₃, citrate, etc. The CP—NPs obtained present amorphous phase which is not affected due to the functionalization process as shown in the examples. The raw nanoparticles may be designed to present a molar ratio Ca/P between 1 and 2.

In the present invention “surface functionalized” may be understood as a core of certain nature (in the present case an inorganic core of calcium phosphate) which is functionalized, i.e. chemical groups are attached on its surface so that they provide different functions to the ones that said core normally presents. For the CP—NPs of the invention, the attached esculetin and euphorbetin molecules provide to the nanoparticles a therapeutic function. The general process of functionalization of the surface of CP—NPs is adding euphorbetin and esculetin (as synthetic molecules or forming part of an ethanolic extract from Euphorbia) to a suspension of CP—NPs and stirring the mixture at room temperature for 24 h in the dark to avoid photolytic decomposition of the bioactive molecules euphorbetin and esculetin.

Preferably, the drug delivery system of the invention is characterized in that the CP—NPs comprise:
a) 0.1 to 7 mg of esculetin per g of the functionalized CP—NPs, more preferably 0.2 to 5 mg of esculetin per g of the functionalized CP—NPs, the most preferred 0.3 mg of esculetin per g of the functionalized CP—NPs and
b) 2 to 10 mg of euphorbetin per g of the functionalized CP—NPs, more preferably 3 to 8 mg of euphorbetin per g of the functionalized CP—NPs, and the most preferred 3.4 mg of euphorbetin per g of the functionalized CP—NPs.

Preferably, in the drug delivery system of the invention, the average size of the CP—NP is between 20 and 50 nm, more preferably 30 to 40 nm.

Another aspect of the invention is the composition as described above or the drug delivery system as described above or the ethanolic extract of Euphorbia as described above for use as a medicament.

Another aspect of the invention is the composition as described above or the drug delivery system as described above or the ethanolic extract of Euphorbia as described above for use in the treatment of cancer, preferably colorectal, pancreatic or glioblastoma.

Another aspect of the invention is a pharmaceutical composition comprising the composition as described above or the drug delivery system as described above or the ethanolic extract of Euphorbia as described above and at least a pharmaceutically acceptable excipient or carrier, and optionally a further active compound.

It is also an aspect of the present invention a pharmaceutical composition, which will be considered a pharmaceutical composition of the present invention, comprising in its formulation the composition of the invention or the extract of the present invention or the drug delivery system of the invention as described above, in any of the possible embodiments described above. For example, that of the extracts obtained by the method of the present invention in which the final evaporation, partial or total, of the ethanol has been carried out. This pharmaceutical composition may be a combined pharmaceutical composition additionally comprising at least one anticancer agent additional to those present in the extract. This anticancer agent may be a commonly used chemotherapeutic compound. In another possible embodiment, also compatible with any other embodiment, the composition may further comprise one or more pharmaceutically acceptable excipients and/or vehicles.

It may also be considered that the above aspect of the invention also implies that it is comprised within the present invention to use the composition of the present invention or the extract of the present invention or the drug delivery system of the present invention as described above for the preparation of a pharmaceutical composition, particularly if the same is intended for the treatment of cancer, especially if the same is selected from the group of colorectal cancer, pancreatic cancer and glioblastoma.

Also, aspects of the invention are the composition of the present invention, or the extract of the present invention, or the drug delivery system of the present invention or the pharmaceutical composition of the present invention as described above, for use in the treatment a type of cancer which is preferably selected from the group of colorectal cancer, pancreatic cancer and glioblastoma. More particularly, the cancer may be selected from the group of colon adenocarcinoma, pancreatic adenocarcinoma and glioblastoma multiforme. In these aspects of the invention, the one referring to the composition, or to the extract or to the drug delivery system or to the pharmaceutical composition, extracts of defatted mature seeds and pharmaceutical compositions prepared therefrom are preferred. Particular preference is given, particularly when starting from defatted seeds to obtain the extract and/or the pharmaceutical formulation, for the treatment of adenocarcinoma of the colon, especially if it is resistant to chemotherapy.

Another aspect of the invention is a method of treatment of cancer comprising administering a therapeutically effective amount of the composition as described above or the drug delivery system as described above, or the pharmaceutical composition as described above to a patient in need thereof.

Aspects referring to the therapeutic use of the composition of the invention, the extract of the invention or the drug delivery system of the invention or the pharmaceutical composition of the invention may also be defined by, or relate to, a method of treating a subject suffering from a cancer which is preferably selected from colorectal cancer, pancreatic cancer and glioblastoma, comprising administering a pharmaceutical composition of the invention or a therapeutically effective amount of the composition of the invention or the extract of the invention or the drug delivery system of the invention. The subject, as in the definition of the composition of the invention, the extract of the invention, the drug delivery system of the invention or the pharmaceutical composition of the invention for therapeutic use, can be any mammal, with preference for a human being.

Thus, the present invention is based on:

1) The development of an ethanolic extract from ripe Euphorbia lathyris seed flour, which, once analyzed, has a high polyphenol content. The extract was obtained from the flour of ripe seeds, without defatting, and from the flour of ripe seeds, previously defatted, after a cold ethanolic extraction process (at a temperature between 0° C. and 8° C., preferably at 4° C.) of short duration (90 min. ) total contact time of ethanol with the flour and the possible pellet resulting from a first extraction process, under nitrogen atmosphere and in acid medium, which is composed mostly of polyphenolic compounds dissolved in a solvent of low toxicity.

2) The potent antitumor activity of Euphorbia lathyris seed extracts when tested on colon cancer cell cultures (T84 of human colorectal cancer, HCT15 of chemotherapy-resistant colorectal cancer) using as control a normal human intestinal epithelial line (CCD18) and tested on the human hepatocyte line HepG2 to determine the therapeutic range. We have also studied the mechanisms of action by which the extracts act on tumor cells to elucidate the molecular pathways that activate cell death.

3) The development of a drug delivery system formed by a core nanoparticles of amorphous calcium phosphate which have been functionalized with the bioactive compounds esculetin and euphorbetin, said molecuels being from a composition including certain concentrations ranges of said molecules or from ethanolic extracts from plants of Euphorbia genus.

The results obtained by the research disclosed in this application demonstrate that:

1) The methodology used for obtaining the ethanolic extract (hydroalcoholic extraction) from the flour from mature, undefatted and defatted seeds of Euphorbia lathyris results in a product very rich in polyphenols.

2) The methodological processing of defatting, which corresponds to preferred embodiments of the present invention, represents an additional advantage of importance for the further development of the ethanolic extract of the present invention, since authors such as Bicchi et al. (2001), as discussed above, have described the presence of diterpenoid fractions corresponding to ingenol and Euphorbia factors in the oil of Euphorbia lathyris. The defatting process will allow, on the one hand, the concentration in the defatted flour of polyphenols of high bioactive capacity to ensure a high extraction yield, and, on the other hand, the elimination of diterpenoid compounds that may be toxic. Even so, as can be seen in Example 2 below in the tests carried out with different cell lines derived from colon cancer, both the extract of the mature seed flour without defatting and of the mature defatted seed have a high antiproliferative activity, the IC50 being very low in both cases, which allows us to consider the use of both extracts for the preparation of pharmaceutical compositions. This idea is reinforced by the fact that both extracts also show antiproliferative capacity against other types of cancer, such as glioblastoma multiforme or pancreatic cancer, so that both extracts, or pharmaceutical compositions prepared from them, could also be used for the treatment of these other two types of cancer.

3) The ethanolic extract of the flour (both undefatted and defatted) of the mature seed of Euphorbia lathyris has a high antitumor activity, specifically in cells derived from colon cancer both resistant and non-resistant to chemotherapy, inducing a potent antiproliferative effect, which was not observed in normal colon epithelium cells or in human hepatocytes, so there is a wide therapeutic range. The results obtained with cells derived from colon cancer are especially significant, not only because of the high incidence of colorectal cancer in the population in general, but also because this is a type of cancer for which no experiments had been carried out with other Euphorbia lathyris extracts or, as in the case of the study by Nam and Lee published in 2000, the results obtained when testing extracts against cells derived from colon cancer had not shown antitumor capacity.

4) The antiproliferative effect of the ethanolic extract from seed flour, both defatted and undefatted, also has a high antitumor activity against glioblastoma multiforme cell lines, one of the most aggressive and difficult to treat cancers, even in assays performed with chemotherapy-resistant cell lines. Antiproliferative effects have also been observed against pancreatic cancer cell lines, specifically pancreatic adenocarcinoma.

5) Non-cytotoxic doses of ethanolic extract from defatted mature seeds of Euphorbia lathyris slow down the migration of colon cancer tumor cells by up to 20% at 72 hours.

6) In the antitumor activity presented by the ethanolic extract, the caspases pathway is involved, producing cell death by apoptosis.

7) Regarding the functionalized CP—NPs of the drug delivery system, the core of CP—NPs does not show toxicity in colon cancer cells (T84) and non-tumor cells (CCD18), but functionalized nanoparticles are able to release the adsorbed esculetin and euphorbetin to colon cancer cells (T84), thus presenting a significant and selective cytotoxicity for colon cancer tumor cells. Furthermore, they show a very low level of hemolysis and a total absence of toxicity in white blood cells.

Thus, the ethanolic extraction methodology provided by the present invention allows obtaining a non-toxic extract for use in biomedicine, particularly as antitumoral. Said methodology represents an enormous advantage for the purpose of its application in patients, since it is based on the use of ethanol, a solvent that is commonly used at low concentrations for the administration of active principles.

The use of ethanol is a great advantage for the clinical application of the extracts obtained, especially when compared to the Euphorbia lathyris seed extracts described in the state of the art in which toxic solvents such as acetone, a compound highly toxic to cells, are used, in addition to including an extraction with ethyl acetate (see Zhang et al., 2017, a paper in which the antitumor capacity and possible toxicity of these extracts are also not studied). Similarly, obtaining Euphorbia lathyris extracts based on the use of methanol (Nam and Lee, 2000) invalidate their use in vivo despite having demonstrated some antitumor capacity. The process of the present invention is also advantageous with respect to those cases in which extraction processes have been tested using a petroleum ether partition after obtaining a 95% Ethanol extract at reflux, after which extracts enriched in terpenes such as the Euphorbia factors are obtained (Duan et al., 2014; Teng et al., 2018; Zhang et al., 2018), unlike the extracts of the present invention, which are enriched in phenolic compounds.

The methodology used in the present invention also has advantages over that used in some papers in which an ethanolic extract is indeed obtained from the seed of Euphorbia lathyris (Meng et al., 2013; Teng et al., 2018). It is important to note that the methodology described in those papers is radically different from the procedure of the present invention, as they use 95% ethanol instead of 50% ethanol and a reflux extraction process for several hours at a non-acidic pH, instead of the short process, with a total extraction time of no more than 1-2 hours, of the procedure of the present invention. Surprisingly, the methodological differences result in the composition of the extracts obtained also being so, it being observed that the variety of compounds present is very different between said extracts and those obtained by the procedure of the present invention. In fact, as discussed above, these authors obtained mainly diterpenoids from the lathyrane-type seed (Euphorbia factors L1, L2, L3, L8 and L9), which do not appear in the extract of the present invention, which mainly presents phenolic compounds, as shown in Example 5 below.

It should also be noted that none of the previous papers, nor others focused on any of the compounds contained in them (such as the paper by Fan et al. of 2019, focused on the L2 factor) mention that the extracts obtained, or the compounds contained in them, show activity against colorectal cancer or cells derived from it, although they do mention cytotoxicity or antiproliferative capacity against cell lines derived from other types of cancers, such as lung and breast cancer, or a very specific type of tumor such as hepatocellular carcinoma. This fact, together with the results of lack of activity of Euphorbia lathyris extracts against cells derived from colon cancer obtained by Nam and Lee in 2000, make it not obvious to expect that ethanolic extracts such as those of the present invention, obtained from mature seeds of Euphorbia lathyris, could present activity against cells derived from colon cancer, as in fact they do, as demonstrated in Example 6 of the present application. Nor was it expected that the ethanolic extracts of the present invention, both from defatted and non defatted seeds, would show activity either against cells derived from glioblastoma or pancreatic cancer, types of cancer for which no antiproliferative assays had been performed with Euphoria lathyris seed extracts.

It is also very remarkable that the antiproliferative effects are observed even in a chemotherapy-resistant colon adenocarcinoma cell line. Moreover, the tumor cell migration assays described in Example 8 showing that, at non-cytotoxic doses of the extract from defatted seeds, colon tumor cells migrate significantly less than controls, indicate that the extract of the present invention, at non-cytotoxic doses, would significantly slow down invasiveness and metastasis formation, a very important factor in the control of any type of cancer, and of colorectal cancer in particular, which further supports the possible use as an anticancer agent, or as a component used in the preparation of anticancer pharmaceutical compositions, of the extracts of the present invention, especially against colon cancer and, in particular, against chemotherapy-resistant colon adenocarcinoma.

As for the composition of the extracts obtained, both the extract from mature, undefatted seeds and the extract from defatted seeds are rich in polyphenols, unlike what is observed in other ethanolic extracts of the prior art. Thus, the ethanolic extracts of the present invention can be defined by biochemical values obtainable for the same according to the results of Example 5 and, generically, can be defined as ethanolic extracts of ripe seeds of Euphobia lathyris rich in polyphenols, as such are the extracts obtainable by the method of the present invention. As can be seen in said Example 5, taking the lower and upper values of the results obtained with both types of seeds, it can be said those that the Euphorbia lathyris extracts of the present invention present a total polyphenol content ranging from 15.64 [15.85-0.21] to 39.31 [33.52+5.79] µg gallic acid equivalents/mg extract), according to the lower values of undefatted seeds and the upper values of defatted seeds obtainable according to the results of Example 5. Similarly, it can be considered that the extracts of the present invention present a reducing capacity ranging from 9.43 [9.71-0.28] to 24.87 [22.95+1.92] µg gallic acid equivalents/mg extract). More specifically, according to the specific data obtained with mature undefatted and defatted seeds, it can be said that the total polyphenol content can be 15.85 ± 0.21 µg gallic acid equivalents/mg extract (mature undefatted seeds) or 33.52 ± 5.79 µg gallic acid equivalents/mg extract (mature defatted seeds) and the reducing capacity can be 9.71 ± 0.28 µg gallic acid equivalents/mg extract (for mature non defatted seeds) or 22.95 ± 1.92 µg gallic acid equivalents/mg extract (for mature defatted seeds), although variations due to possible differences in yield and used seed batches mean that these values have to be interpreted as indicative.

As for the compounds present, the presence of esculetin, euphorbetin, gaultherin, nicotiflorin (Kaempferol-3-ruthinoside) and carnosol stands out. An extract of the present invention should contain at least one of them. As previously discussed in the “Background of the Invention” section, these are polyphenols that have previously been related to antitumor activity, but in assays performed with the compounds independently, obtained from commercial houses, they have been related in previous studies to antitumor activity (Lee et al., 2017 and 2019; Turkekul et al., 2018; Aliebrahimi et al., 2018; HefnyGad et al., 2018). Thus, it is relevant to highlight, for example, the studies reported by Lee et al. (2017), which have not been performed with extracts of Euphorbia lathyris, but with the esculetin molecule, which implies that the observed activity does not correlate with the possible interaction between the different compounds detected in the extract of the present invention, an interaction that may modify the effectiveness of the biological effect of the preparations in which said extract is included, thus being unpredictable its activity.

As can be seen in Example 5, the major components are esculetin, kaempferol-3-ruthinoside (nicotiflorin) and euphorbetin. The presence in an ethanolic extract of mature seeds of Euphorbia lathyris of at least two of these compounds or, preferably, all three, particularly if gaultherin and carnasol are also present, and most especially if esculetin and/or kaempferol-3-rutoside is present in a concentration in the ranges defined by the minimum and maximum values in Table 4 or the values in Table 5 (i.e., for esculetin, according to Table 4 the ranges from 985.9 µg/L to 1197.2 µg/L and 2041.5 µg/L to 2325.8 µg/L wt. of compound : extract volume, all values inclusive, or according to Table 5, 0.4 ± 0.05 mg compound per 100 mg extract or 0.21 ± 0.023 mg compound per 100 mg extract and, for kaempferol-3-ruthinoside, according to Table 4, the ranges from 59.8 µg/L to 61.1 µg/L and from 188.3 µg/Lto 354 µg/L, compound weight: extract volume, all values inclusive, or, according to Table 5, 0.02 ± 0.003 mg compound per 100 mg extract or 0.21 ± 0.023 mg compound per 100 mg extract.

For any of the above-mentioned possibilities of definition of the extract, the option corresponding to the extract obtainable from defatted seeds is always preferred, and particularly, the combination of all of them.

As can be seen in Example 5, the definitions of the ethanolic extract of the present invention indicated above correspond to values obtained by applying to mature seeds of Euphorbia lathyris the method of the present invention, which is preferred for the extracts of the present invention, particularly for their possible therapeutic use. Extracts obtained from seeds defatted by mechanical cold pressing under the conditions applied to obtain the extracts obtained in Example 5 are preferred, particularly if the seed milling and extraction conditions used in said Example 5 have also been applied, including additional extraction.

As discussed above, the extracts of the present invention show antiproliferative effects against colorectal cancer, pancreatic cancer and glioblastoma cell lines, as can be seen in Examples 6 to 8 of the present application, carried out on colon adenocarcinoma, pancreatic adenocarcinoma and glioblastoma multiforme, in which it can also be seen that the toxicity values found allow considering the use of extracts obtained from mature seeds of Euphorbia lathyris, both non defatted and defatted, although the latter are preferred, especially for colon adenocarcinoma, even if it is resistant to chemotherapy, as can be seen in Example 8.

Thus, the extracts of the present invention may form part of the formulation of pharmaceutical compositions, optionally having previously evaporated part or all of the ethanol from the extract, compositions which may have the same above-mentioned therapeutic uses for the extracts. Said compositions may comprise pharmaceutically acceptable excipients and/or vehicles and/or be combined pharmaceutical compositions which additionally comprise at least one anti-cancer agent additional to those present in the extract. Said pharmaceutical compositions may be for the same uses as mentioned above for the extracts.

The invention, its features and possible embodiments thereof are explained in greater detail and are supplemented by the Examples and Figures below, which are included for illustrative and non-limiting purposes.

### BRIEF DESCRIPTION OF FIGURES

FIG. 1. UPLC-ESI(-)-MS total ion current chromatogram of Euphorbia extract (A). 1 = esculetin; 2 = euphorbetin. The chemical structures of the phytochemicals are also shown. UV-vis spectra (B) of Euphorbia extract and a solution containing equal concentrations of standards of esculetin and euphorbetin.

FIG. 2. XRD diffractograms (A) and FTIR spectra (B) of CP—NP and BC—CP—NP.

FIG. 3. TEM images of CP—NP (A), BC—CP—NP (B).

FIG. 4. UPLC-ESI(-)-MS total ion current chromatogram of the solution resulting from the partial dissolution of BC—CP—NP (A). Retention times corresponding to molecular weight of euphorbetin and esculetin are shown in B and C (1 = esculetin; 2 = euphorbetin).

FIG. 5. BC release profiles from amorphous BC—CP—NP in Potassium Phosphate Buffer pH 7.4 (empty squares) and in Sodium Citrate Buffer pH 5.5 (empty circles) during 9 days. The experimental data were fitted by first order kinetic models to obtain the theoretical release curves represented by dashed lines (release constants, k, are shown in brackets).

FIG. 6. Antiproliferative activity of BC—CP—NP and CP—NP in colon cells. T84 (A) and CCD18 (B) cells were exposed to nanoparticles at different concentrations (µg nanoparticle/ml) for 72 h to obtain the Ic50 values (C). Data are presented as the mean ± standard deviation of three independent experiments; *P<0.05 vs respective control group. **P<0.01 vs respective control group.

FIG. 7. Antiproliferative assays of the Esculetin and Euphorbetin compounds. T84 cells were exposed to Esculetin (A) and Euphorbetin (B) for 72 h in order to determine the Ic50 values (C). Data are presented as the mean ± standard deviation of three independent experiments; *P<0.05 vs respective control group. **P<0.01 vs respective control group.

FIG. 8. Antiproliferative assay of the combination of Esculetin and Euphorbetin. T84 cells were exposed to different Esculetin and Euphorbetin combinations for 72 h in order to determine the Ic50 values and synergic effect when they are combined. Data are presented as the mean ± standard deviation of three independent experiments.

FIG. 9. Blood biocompatibility assay of nanoparticles. (A) Representative Images of optical microscopy of erythrocytes after treatment with different concentrations of BC—CP—NP and CP—NP. Scale bar 50 µm. (B) Hemolysis capacity was expressed as the percentage of erythrocytes lysates versus concentration of nanoparticles (µg/ml). (C) Proliferation assay of white blood cells when they were treated with different concentrations of BC—CP—NP and CP—NP during 1 h and 12 h. Data represent the mean value ± SD of triplicate cultures

FIG. 10. Shows an outline of the methodology for obtaining an ethanolic extract from seed flour.

FIG. 11. Shows a representative image of the macroscopic difference between ethanolic extracts of undefatted mature Euphorbia lathyris seed flour (a), and previously defatted mature Euphorbia lathyris seed flour (b) based on the different presence of lipid compounds.

FIG. 12. Shows a plot of the chromatography replicates of the ethanolic extracts from the undefatted mature seed flour of Euphorbia lathyris.

FIG. 13. Shows a plot of the chromatographic replicates of ethanolic extracts from previously defatted mature seed flour of Euphorbia lathyris.

FIG. 14. Shows Western Blot membrane reveal for Caspase 3, 8 and 9 expression in colon tumor line T84 cells treated with an IC50 of the ethanolic extract from defatted mature seed flour of Euphorbia lathyris.

FIG. 15. Shows a representation of the expression level of Caspases 3, 8 and 9 in T84 colon tumor cells treated with an Ic50 of the ethanolic extract from defatted mature seed flour of Euphorbia lathyris.

FIG. 16. Shows images obtained at different times (0, 8, 24, 24, 48 and 72 hours) of cell migration assays performed with subcytotoxic doses of ethanolic extract of defatted mature seed flour of Euphorbia lathyris on T84 colon tumor cells.

FIG. 17. Shown, a representation of the percentage of cell migration upon treatment with a subcytotoxic dose of ethanolic extract of defatted mature seed flour of Euphorbia lathyris T84 colon tumor cells at different times.

### EXAMPLES

### Materials and Methods

### Extraction and Characterization of Euphorbia Extract

Mature seeds of Euphorbia lathyris S3201 were obtained by the seed defatting process of the invention, by separating of the oleaginous part of the mature seed by means of a cold seed oil extraction press without exceeding 40° C. The average working speed of the extraction process was 2-3 kg seed/h, and the average extraction yield ranged 15-25%. As a result of the former processing conditions, a defatted flour with a particle size of 100-150 µm was obtained. Defatted flour samples were stored at -20° C. in absence of light until use. Defatted flour from mature seed of Euphorbia lathyris were used to obtain a polyphenol-rich ethanolic extract. This process was developed mixing 5 g of defatted flour with 15 ml of Ethanol:H2O:HCl(37% w/w) solution (50:50:0.25) at pH 2 and 4° C. in a reducing atmosphere (with nitrogen) for 30 minutes in a magnetic stirrer. After 30 minutes stirring, the extract was centrifuged at 3.000 rpm for 5 minutes. The supernatant was stored, and the pellet recovered to repeat the process. Finally, all the supernatants were mixed and stored at -20° C. After 24 hours, the extracts were centrifuged at 3.000 rpm for 5 minutes and supernatant collected. To determine the ethanolic extract yield and concentration, aliquots (1 mL) were obtained, and ethanol was evaporated using a vacuum evaporator. The evaporated extracts were frozen in liquid nitrogen and lyophilized during 24 hours. Then, the extract dry weight was calculated by difference with the container containing each aliquot which was referred to a volume of 1 mL of initial extract, to the total volume of extract obtained, and finally to the grams of flour.

Identification of bioactive compounds that are part of the Euphorbia lathyris extract was achieved by means of ultra-high performance liquid chromatography-electrospray ionization tandem mass spectroscopy (UPLC-ESI(-)-MS). The chromatographic method was the same as described for identification of bioactive compounds adsorption on nanoparticles. The identification of major active ingredients from Euphorbia extract was based on their retention times (RT) and mass (MS) fragments. Besides the UPLC-MS analysis, Euphorbia extract was characterized by UV-vis spectroscopy. The UV-vis spectra of the most interesting bioactive compounds, esculetin and euphorbetin, exhibited an absorbance band-centred at 344 nm (ε³⁴⁴=60.2 mg mL⁻¹ cm⁻¹).

Ultra-high-performance liquid chromatography coupled to a Diode array detection (UPLC-DAD) was employed for quantifying bioactive compounds from Euphorbia extract. Plant extract was filtered through 0.22 µm nylon disk filters and 10 µL of filtered extract was injected into the chromatograph. Analytical separation was carried out in the same conditions as the quantification of bioactive compounds adsorption on nanoparticles.

### Synthesis of Biomimetic Nanoparticles

CP-NP nanoparticles were synthesized following a precipitation method previously reported with modifications (Delgado-López et al., 2012, (WO2016012452A1/en). Two solutions (1:1 v/v, 100 mL total) of (a) 0.12 M K₂HPO₄ + 0.1 M Na₂CO₃ and (b) 0.2 M CaCl₂ + 0.2 M Na₃(cit) were mixed and the resulting aqueous solution became milky. The mixed solution was continuously stirred at around 250 rpm using a stirring hot plate for about 5 min at room temperature. Afterwards, the nanoparticles were repeatedly washed with ultrapure water by centrifugation to remove unreacted salts and dried for further characterizations. Under these conditions the amorphous calcium phosphate nanoparticles were obtained.

### CP-NP Functionalization

CP-NP nanoparticles (100 mg) were suspended in 5 mL of ultrapure water and sonicated for 30 min. 100 mg of Euphorbia extract was added to the nanoparticles suspension. The mixture was stirred at room temperature for 24 h in the dark to avoid photolytic decomposition of bioactive compounds (BC) in the extract. Subsequently, bioactive compound-loaded CP—NP nanoparticles (BC—CP—NP) were separated from unbound compounds by centrifugation at 10.000 rpm for 5 min. Afterward BC—CP—NP were carefully washed three times with 10 mL of ultrapure water to remove the physically adsorbed bioactive molecules.

### Characterization of the Nanoparticles

Fourier transform infrared (FTIR) spectra were recorded on a FTIR spectrometer using the KBr pellet method. Each pellet was prepared by mixing approximately 3 mg of powdered sample with approx. 200 mg of anhydrous KBr and pressed into 7 mm diameter discs. Pure KBr discs were used as background. FTIR spectra in transmittance mode were registered from 4000 cm⁻¹ to 400 cm⁻¹ with a resolution of 4 cm⁻¹. Powder X-ray diffraction (PXRD) patterns of the samples were collected using a Bruker D8 Advance diffractometer equipped with a Lynx-eye position sensitive detector using Cu Kα radiation (λ = 1.54178 Å) generated at 40 kV and 40 mA. Diffractograms were recorded in the 2θ range from 15 to 70° with a step size (2θ) of 0.02 and a counting time of 1 s. Transmission electron microscopy (TEM) analyses were performed with a Carl Zeiss SMT LIBRA 120 PLUS microscope operating at 120 kV. The powder samples were ultrasonically dispersed in ultrapure water using an Allendale-Ultrasonic cleaner and then few droplets of the slurry were deposited on mesh copper TEM grids covered with thin amorphous carbon films and incubated for several minutes.

### Identification and Quantification of Bioactive Compounds Adsorbed on Nanoparticles

The identification of the bioactive compounds adsorbed on the nanoparticles was carried out by Ultra-high Performance Liquid Chromatography tandem orthogonal acceleration time-of-flight mass spectrometer with an electrospray-ionization technique (UPLC-ESI(-)-MS). BC—CP—NP (1 mg) were partially decomposed in nitric acid (pH=3) by stirring during 24 h. Partial dissolution of BC—CP—NP ensures the release of adsorbed molecules. 10 µL of the final dissolution was filtered through 0.22 µm nylon disk filters and injected into the chromatograph. Analytical separation of bioactive compounds was performed on a C18 column (100 mm x 2.1 mm internal diameter, 1.6 µm) at room temperature. A mobile phase consisting in a gradient program combining deionized water with 0.5% of acetic acid as solvent A and acetonitrile as solvent B was used. The initial conditions were 90% A and 10% B. A linear gradient was then established to reach 100% (v/v) of B at 5 min. Total run time was 8 minutes. Mobile phase flow rate was 0.3 mL min⁻¹. After chromatographic separation, a high-resolution mass spectrometry analysis was carried out in negative electrospray ionization. The gas used for desolvation (500 L h⁻¹) and cone (50 L h⁻¹) was high-purity nitrogen. Spectra were recorded over the mass/charge (m/z) range of 100-1200. All the compounds were identified based on their retention times (RT) and mass (MS) fragments. Based on these data, the compounds were tentatively identified using a specific software.

The analytical quantification of the adsorbed bioactive compounds was performed by Ultra-Performance Liquid Chromatography coupled to a Diode Array Detection (UPLC-DAD). BC—CP—NP (1 mg) were partially dissolved in an acidic solution (pH=3) by stirring during 24 h to ensure complete release of adsorbed bioactive compounds. After filtering through 0.22 µm nylon disk filters, analytical separation of bioactive compounds was performed as described for identification of bioactive compounds adsorption on nanoparticles. The concentrations of BC were evaluated from peak areas at 344 nm, using calibration curves established with the corresponding standards, esculetin and euphorbetin. Once, the amounts of adsorbed BC were measured, the loading capacity (LC) was calculated as follows:

$$L C = w e i g h t o f a b s o r b e d B C \left(m g\right) w e i g h t o f B C - A C P \left(g\right)$$

LC represents the mass of bioactive molecules adsorbed per unit mass of BC—CP—NP (mg g-1).

### Release of Bioactive Compounds From BC—CP—NP

The time-dependent release of BC from BC—CP—NP was analyzed at two physiological pH conditions. At a pH of 7.4, the physiological pH of blood and at a pH of 5.5, simulating the pH inside cell lysosomes (pH~5) (Feng et al., 2018). 15 mg of BC—CP—NP were immersed in Potasium Phosphate Buffer (10 mM, 3 mL, pH 7.4) and in Sodium Citrate Buffer (10 mM, 3 mL, pH 5.5), respectively, at room temperature. UV-Vis spectra of the suspensions were recorded every 30 min during 9 days. The UV-vis spectra of the most interesting bioactive compounds, esculetin and euphorbetin, exhibited an absorbance band-centred at 348 nm in Sodium Citrate Buffer (pH 5.5) and at 366 nm in Potasium Phosphate Buffer (pH 7.4). After 9 days, the release of BC from BC—CP—NP was complete.

### Example 1 Extraction and Characterization of Euphorbia Extract

Previously reported analysis of other crude extracts of the genus Euphorbia showed the presence of two molecules of great medicinal interest, named esculetin and euphorbetin. Both compounds (insets FIG. 1A) are structurally-related coumarin derivatives, the esculetin (C₉H₆O₄) is the monomeric coumarin of the euphorbetin (C₁₈H₁₀O₈), its dimer. UPLC-ESI(-)-MS total ion current chromatogram (FIG. 1A) showed that Euphorbia extract contained, among others, high concentrations of both phytochemicals. The retention time of 4.174 min, 4.665 and 5.014 min corresponds to the molecular weight of esculetin and euphorbetin, respectively (labelled as 1 and 2 in FIG. 1A). Coumarin derivatives were indeed unambiguously identified in Euphorbia extract by the comparison of their retention times to reference standards (Table 1). Simultaneous quantitative analysis of the two compounds was accomplished by UPLC-DAD. Using external standards of esculetin and euphorbetin, the concentration of the coumarin derivatives in the Euphorbia extract was determined as assessed by their retention times (RT) and absorption band at 344 nm. The contents of esculetin and euphorbetin in the Euphorbia extract were 19.2 mg g⁻¹ and 7 mg g⁻¹, respectively.

The Euphorbia extract was also analysed by UV-vis spectroscopy (FIG. 1B). The UV-vis spectrum showed a main absorbance band centred at 344 nm, which perfectly matches with the spectrum of an aqueous ethanolic solution containing both standards, esculetin and euphorbetin, at the same concentrations as in the Euphorbia extract. (FIG. 1B). Thus, the absorbance at 344 nm of the Euphorbia extract was the result of the joint contribution of esculetin and euphorbetin.

TABLE 1

Identification of esculetin and euphorbetin from Euphorbia extract.

| Retention Time (min) | Compound (molecular formula) | Molecular mass [M-H]⁻ (g mol⁻¹) | PPM |
|---|---|---|---|
| 4.10 | Esculetin (C₉H₅O₄) | 177.018 | -6.2 |
| 4.63 | Euphorbetin (C₁₈H₉O₆) | 353.03 | 2.8 |

PPM: Difference between the observed mass and the calculated mass.

### Example 2 Synthesis of Nanoparticles Functionalized With Bioactive Species of the Euphorbia Extract

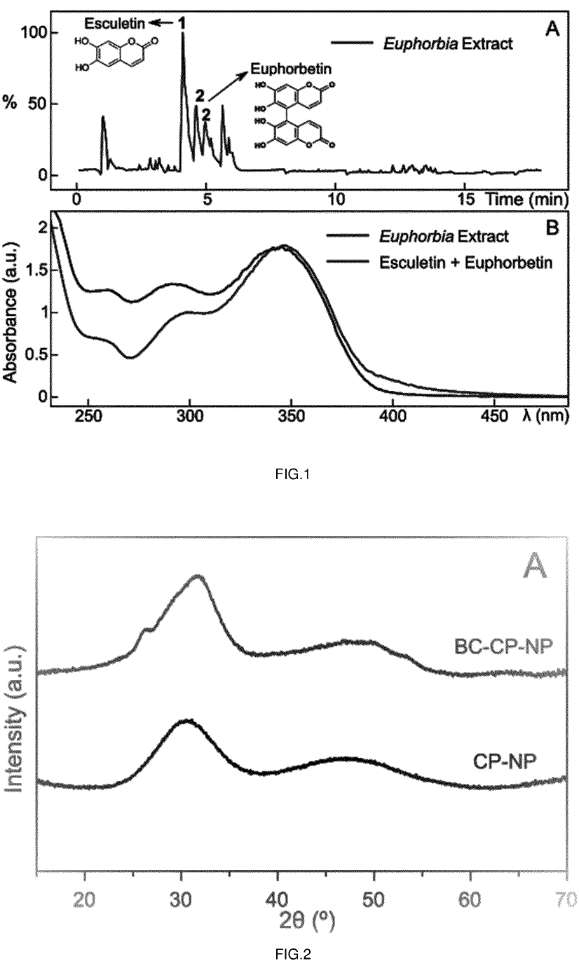

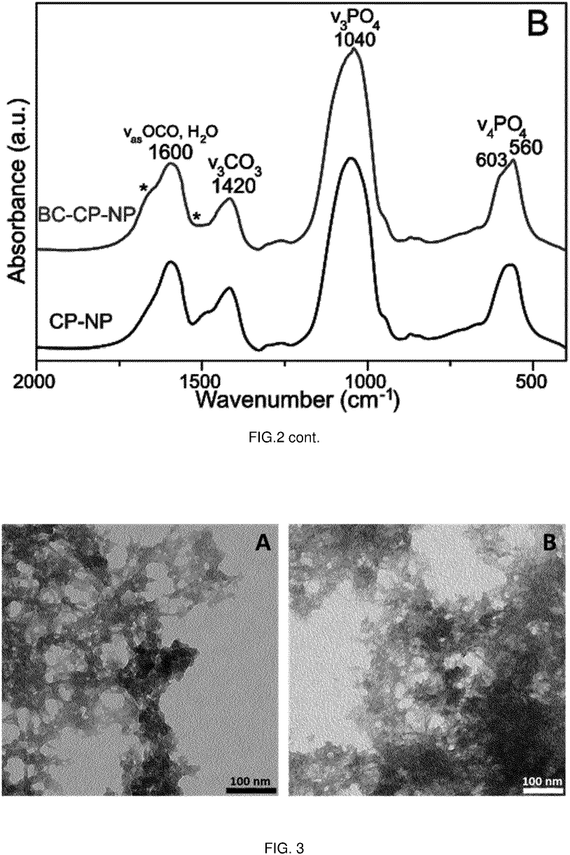

The precipitation method was designed to obtain biomimetic CP—NP nanoparticles whose composition -including citrate and carbonate ions- mimics the mineral phase of bones. The XRD pattern of native CP—NP showed two broad humps, indicating the lack of long-range periodicity typical of calcium phosphate amorphous phases (FIG. 2A). During the adsorption, CP—NP phase started to crystallize as indicated by the incipient diffraction peak at 25.8 degrees (FIG. 2A) related to the 002 reflection of nanocrystalline apatite.

Nanoparticles were also analysed by FTIR spectroscopy. FIG. 2B represents the FTIR spectra of CP-NP and BC—CP—NP. FTIR spectrum of CP—NP displays broad bands characteristic of the amorphous nature. Interestingly, BC—CP—NP FTIR spectrum displays two absorption bands at ca. 1510 cm⁻¹ and 1640 cm⁻¹ (marked with *) that are associated respectively, to the functional groups C═C aromatic and C═C alkene of the adsorbed bioactive compounds on nanoparticles surface. The change of the nanoparticles colour from white CP—NP to yellowish BC—CP—NP—yellowish being similar to the plant extract colour- confirms the effective adsorption (data not shown).

TEM images of CP—NP (FIG. 3A) revealed aggregates of round-shaped amorphous nanoparticles with morphological features previously observed in nanoparticles obtained using the same synthesis route. TEM images of BC—CP—NP (FIG. 3B) showed very similar amorphous nanoparticles as pointed out by the XRD pattern (FIG. 2A). After functionalization, BC—CP—NP morphology remained practically unchanged.

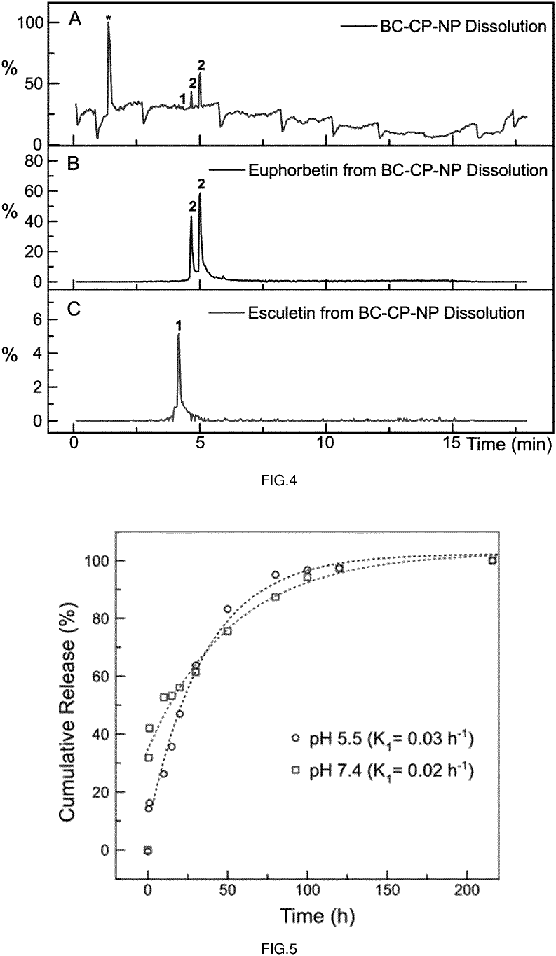

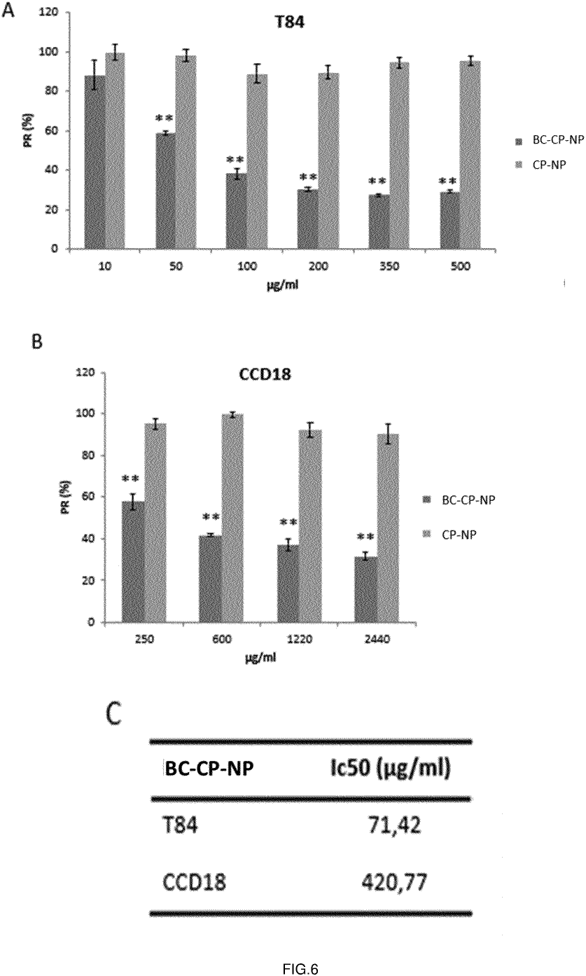

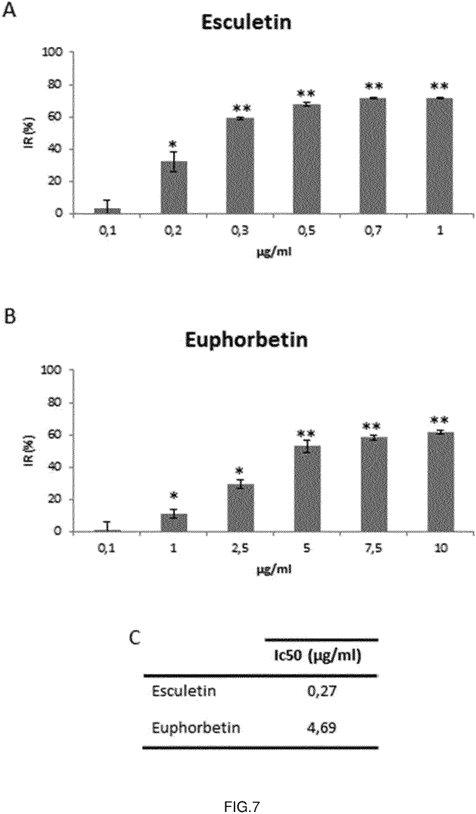

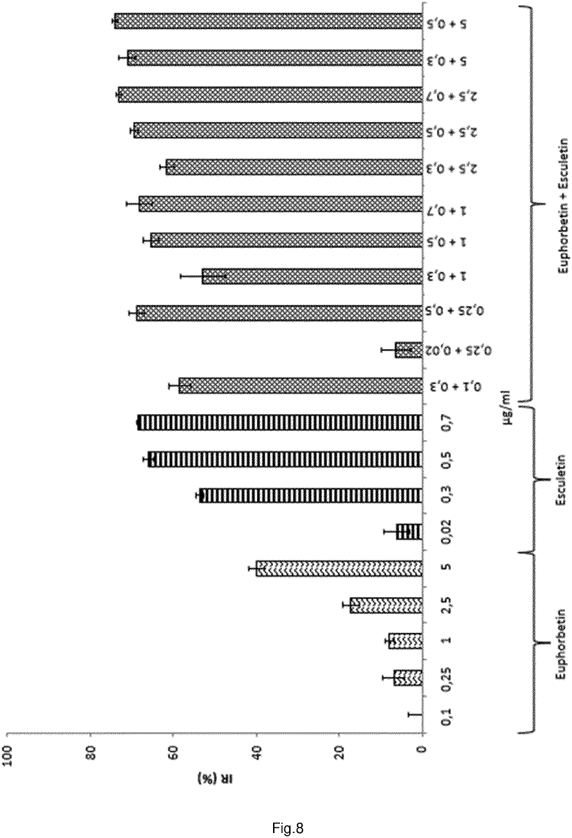

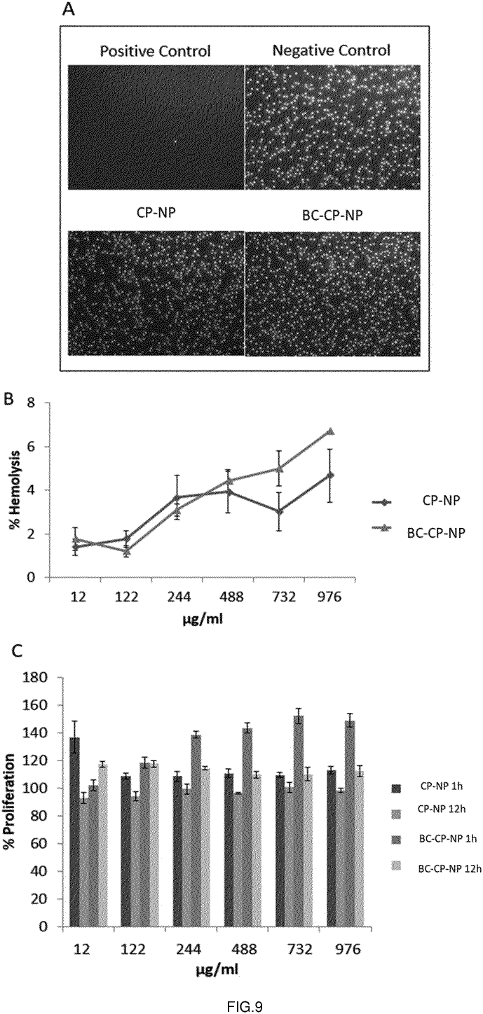

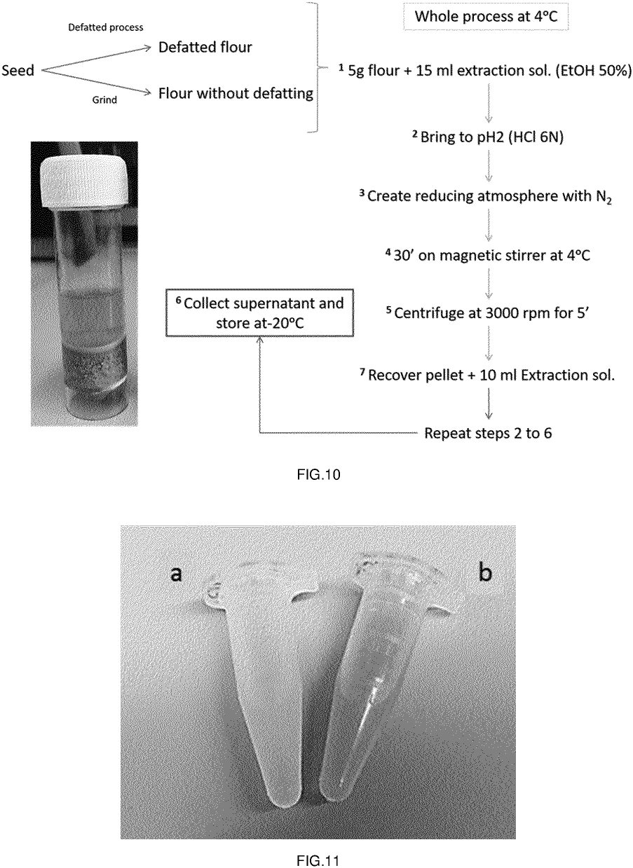

FIG. 4 shows UPLC-ESI(-)-MS total ion current chromatograms of BC—CP—NP dissolution (A-C). Both bioactive compounds, esculetin and euphorbetin, were unambiguously identified once desorption of BC from the surface of BC—CP—NP occurred (FIG. 4A). In BC—CP—NP dissolution chromatogram, the intensity of the peak of euphorbetin (FIG. 4A, labelled as 2) is higher than esculetin peak (labelled as 1) suggesting higher concentration of euphorbetin after desorption.

Mass spectrometry qualitative analysis confirmed the specific adsorption of both molecules from Euphorbia extract (esculetin and euphorbetin) on BC—CP—NP surface. Furthermore, we assessed the mayor peak in FIG. 4B at 1.378 min, which corresponds to citrate (labelled as *), since the mass fragment spectrum indicated a molecular weight of 191.124 g mol⁻¹. Upon particle dissolution, citrate was desorbed as the rest of molecules. The adsorption of citrate (used during the synthesis) on CP—NP is well reported.

Quantitative analysis of the two coumarins from Euphorbia extract adsorbed on BC—CP—NP was accomplished by UPLC-DAD. Equation 1 was used to find out LC for both coumarin compounds, esculetin and euphorbetin. Results are listed in Table 3. LC were 0.3 mg of esculetin and 3.4 mg of euphorbetin per gram of BC—CP—NP nanoparticles. This is in line with the intensity ratio of the peaks of esculetin and euphorbetin previously depicted in FIG. 4B, which suggested a much higher loading of euphorbetin on BC—CP—NP surface than esculetin.

TABLE 2

BC-CP-NP Loading capacity (LC) of the two coumarin derivatives.

| Compound | LC (mg g⁻¹) |
|---|---|
| Esculetin | 0.3 ± 01.1 |
| Euphorbetin | 3.4 ± 0.9 |

### Example 3. Slow and Gradual Release of BC

The pH effect on BC release kinetic was analyzed to assess the use of BC—CP—NP as a controlled drug delivery system, since pH-dependent dissolution of BC—CP—NP can result in different release profiles depending on the pH of different body fluids. The physiological pH of blood is close to 7.4, whilst the pH of the tumor microenvironment is more acidic (pH~6.5) and the pH inside cell lysosomes is around 5.0. Thus, we evaluated BC release kinetic at a pH of 7.4 (physiological pH of blood) and at a more acidic pH of 5.5 (pH value between the pH of tumor microenvironment and the pH inside cell lysosomes). FIG. 5 displays the time-dependent BC release from BC—CP—NP nanoparticles, during 9 days. The initial burst effect can be due to the desorption of weakly bound BC at the CP—NP surface. For both pH conditions, BC then follows a gradual and slow-release profile, which can be fitted to first order kinetics with release rates in the range 0.02 h⁻¹ < k < 0.03 h⁻¹.

Calcium phosphates exhibit a pH-dependent solubility, i.e., the lower the pH, the higher is the solubility. This behavior can explain the slightly higher release at the more acidic pH 5.5 (k(pH 5.5) = 0.03 h⁻¹; k(pH 7.4) = 0.02 h⁻¹). These kinetics profiles are in agreement with the constants of nanoparticles dissolution (Ramírez-Rodríguez, 2020). This means that the adsorbed species are released upon the slow particle dissolution occurring in the aqueous media, slightly faster at the more acidic pH 5.5.

### Example 4. In Vitro Biological Assays

The human colon adenocarcinoma cell line T84was purchased from the American Type Culture Collection (Rockville, MD, USA). The non-tumor colon cell line CCD18 (human colon epithelial cell line) was used as control. All cell lines were grown in Dulbecco’s Modified Eagle’s Medium (DMEM) supplemented with 10% heat-inactivated fetal bovine serum (FBS) and ATB (antibiotic, streptomycin + amphotericin B) at 1% and maintained in an incubator at 37° C. and 5% CO₂ humidified atmosphere.

Cells were seeded in 48-well plates with DMEM (Dulbecco’s modified Eagles Medium) (300 ul) at a density of 4 x 10³ cells/well in CCD18 and 5 x 10³ cells/well in T84. After 24 hours, cell cultures were exposed 72 h to increasing concentrations of the nanoparticles dissolved in DMEM. In addition, bioactive components of the extract, Esculetin (range from 0.1 µg/ml to 5 µg/ml) and Euphorbetin (range from 0.02 µg/ml to 0.7 µg/ml), were combined at different concentration in order to determine their antitumor activity and to compare with nanoparticle activity and ethanolic extract activity. After incubation time, cells were fixed with trichloroacetic acid (TCA) at 10% for 20 minutes at 4° C. Once dried, the plates were stained with 0.4% sulforhodamine B (SRB) in 1% of acetic acid (20 min, in agitation). After three washes with 1% of acetic acid, the SRB was solubilized with Trizma® (10 mM, pH 10.5). Finally, the optical density (OD) at 492 nm was measured in a spectrophotometer. Cell survival (%) was calculated according to the following equation:

$$C e l l S u r v i v a l \left(\%\right) = \frac{T r e a t e d c e l l s O D - b l a n k}{C o n t r o l O D - b l a n k} x 100$$

A hemolysis assay was carried out following a modified version of the protocol reported by Leiva et al., 2017. Briefly, human blood (25 mL) from a healthy donor transferred by Andalusian Public Health System Biobank was recovered into collection tubes with EDTA and centrifuged (500×g for 5 min). The plasma was discarded, and the erythrocytes were washed with 150 mM NaCl, mixed by inversion, and centrifuged at 500×g for 5 min (twice). The supernatant was then aspirated and replaced with phosphate buffered saline (PBS) at pH 7.4. The erythrocytes were diluted (1:50), and 190 µL of the diluted erythrocytes (pH 7.4) was added to each well of a V-bottomed 96-well plate. BC—CP—NP and CP—NP dissolved in DMEM at different concentrations (dose range from 1 µg/mL to 980 µg/mL) were added in a volume of 10 µL per well. Positive and negative controls were 20% Triton X-100 (10 µL) and phosphate buffer pH 7.4 (10 µL), respectively. The plate was incubated for 1 h at 37° C. under stirring (15 rpm), centrifuged at 500×g for 5 min, and then 100 mL of the supernatant was transferred into a flat-bottomed 96-well plate. The percentage of hemoglobin released from the erythrocytes was determined at a wavelength of 492 nm using a colorimeter. This assay was performed in triplicate and the hemolysis percentage was calculated using the following formula:

$$\begin{matrix}H e m o l y s i s \left(\%\right) = \\ \frac{a b s . o f t h e s a m p l e - a b s . o f t h e n e g a t i v e c o n t r o l}{a b s . o f t h e p o s i t i v e c o n t r o l} x 100\end{matrix}$$

Optical microscopy images of the erythrocytes treated with the different formulations were taken at the highest dose used (500 µg/ml) to analyze the morphological modifications.

In addition, white blood cells (WBCs) were isolated and assayed by nanoparticle toxicity. For its isolation, blood was poured into Ficoll-Paque (v/v) avoiding mixing. It was centrifuged at 400 g for 30 minutes. The upper layer of blood plasma was removed and the middle layer containing the white cells was taken. Then, cells were diluted in 10 ml of PBS and centrifuged again at 1000 g for 10 minutes. Finally, after pulling out the supernatant, the cells were resuspended in RPMI 1640 supplemented with 10% FBS and 1% antibiotic. These cells were cultured in 96-well plates at a density of 2 × 10⁴ cells/well in a volume of 90 µL of which 10 µL of BC—CP—NP and CP—NP were added to each well from stock solutions of different concentration to reach a final concentration from 1 to 980 µg/mL. The treatments were incubated for 1 and 12 h at 37° C. and 5% CO₂ in a humidified atmosphere, after which the viability of the WBCs was determined by the Cell Counting Kit-8 (CCK-8). This viability assay consists in adding to each well CCK-8 solution directly to the cells, incubate the plate for 3 hours and measure the absorbance at 450 nm using a microplate reader.

Nanoparticles were assayed in colon cancer cells (T84) and non-tumor cells (CCD18). As shown in FIG. 5, in both lines no toxicity is observed for the CP—NP nanoparticles. However, BC—CP—NP showed a great antitumor effect in the T84 cell line with an IC₅₀ value of 71.42 µg/ml. In contrast, the IC₅₀ value in the non-tumor cell line CCD18was 420.77 µg/ml. Thus, BC—CP—NP exhibited a significant and selective cytotoxicity for colon cancer tumor cells.

In order to compare antitumor activity of the nanoparticle to esculetin/euphorbetin in the ethanolic extract of Euphorbia lathyris, combinations of the isolated compounds were carried out. First, values of IC50 of each compound (Esculetin and Euphorbetin) were obtained (FIG. 5), being Esculetin the compound with a higher antiproliferative activity (0.27 µg/ml). To carry out the combinations of euphorbetin and esculetin, doses lower than the IC50 of each compound were combined. As shown in FIG. 6, the same euphorbetin/esculetin combination (0.3/0.1 µg/ml) as that found in the extract did not modulate IC50 value. However, when euphorbetin/esculetin were combined using the same nanoparticle concentrations (0.25/0.02 µg/ml) only a very low percentage (10%) of cellular proliferation was inhibited. Therefore, the use of the nanoparticle associated to euphorbetin/esculetin represented a very notable antitumor action advantage in relation to the independent extract.

In order to evaluate the blood cell toxicity of nanoparticles, a hemolysis test was performed using human erythrocytes. As shown in FIG. 9A and B, none of the nanoparticles caused erythrocytes agglutination or modification of their morphology. A very low level of hemolysis (around 2%) was detected at doses of 122 mg/ml. In addition, white cell toxicity of nanoparticles was also tested. As shown in FIG. 9C, nanoparticles showed a total absence of toxicity in white blood cells (viability around 100%) after 1 and 12 h of exposure at all doses tested.

### Example 5 Methodology for Obtaining the Ethanolic Extract and Analysis

The method of obtaining the ethanolic extract that was developed from the flour of the mature seed of Euphorbia lathyris, both undefatted and defatted, is schematized in FIG. 5 and was as follows:

1. Depending on the case:

(a) For the case of flour without defatting: Grind the ripe seeds obtaining flour, which is stored at -20° C.

(b) In the case of defatted flour: Separation of the oleaginous part of the mature seed from the solid part or pellet is carried out using a seed oil extraction press, KOMET series, which is characterized by its special cold pressing process in which, instead of individual compression screws, screw conveyors are used to squeeze the oil. In this machine the oilseeds are gently pressed without exceeding 50° C. With an average working speed of 2-3 kg of seed per hour and an oil-to-seed conversion yield between 15-25%. As a result, a compact and defatted dry cake is obtained. This cake is then milled at 28,000 rpm to obtain a defatted flour with a particle size of between 100 and 150 microns, which is then stored at -20° C. 2. Weigh 5 g. of defatted flour in a beaker and immediately put the beaker on ice to work at a temperature of 4° C.

3. Add 15 ml of extraction solution (50% ethanol (EtOH) = 50 ml EtOH + 50 ml double distilled water + 0.2 ml hydrochloric acid (HCl).

4. Shake on a magnetic stirrer at 300 rpm.

5. Take to pH 2 (adding HCl 6N).

6. Add gaseous nitrogen so that there is no oxidizing environment and the polyphenols are properly preserved.

7. Leave 30′ in agitation to 4° C.

8. Centrifuge to 7000 rpm during 5′ at 4° C.

9. Collect the supernatant and preserve it at -20° C.

10. The precipitate is resuspended in 10 ml of extraction solution and a second extraction is performed following the steps indicated above.

11. Finally, after performing the pertinent extractions, the supernatants of each extraction are pooled and stored at -20° C. until use.

12. The pellet originated in the last centrifuge is discarded.

Following the described extraction protocol, ethanolic extracts were obtained from the flour of the mature, undefatted and defatted seeds of Euphorbia lathyris. The objective of this double process was to compare the activity of the extracts by removing the high percentage of lipid compounds from the mature seed without defatting (see FIG. 11, where the macroscopic difference between both ethanolic extracts can be appreciated based on the different presence of lipid compounds) and to compare their functional activity against the extracts obtained from previously defatted mature seed flour. Table 3 shows the yield, antioxidant activity (reducing capacity) and total polyphenols of the extract from mature, undefatted and previously defatted Euphorbia lathyris seed flour.

To determine the yield, the ethanolic extract was divided into 1 mL aliquots for ethanol evaporation and subsequent lyophilization of the remaining water in the extract. To remove ethanol from the ethanolic extracts, ethanol was evaporated under vacuum using a Savant DNA 120 evaporation system (Thermo Scientific) for 60 min. After ethanol evaporation, the aliquots with the remaining extract were frozen in liquid nitrogen and lyophilized using a TELSTAR Cryodos-50 lyophilizer where they were kept for 24 hours. After lyophilization, the dry weight of the extract was calculated by difference with the container containing each aliquot and this dry weight was referred to a volume of 1 mL of initial extract, then to the total volume of extract obtained, and finally to the grams of seed flour used to prepare the extract.

Total polyphenols were determined by the technique of Dewanto et al. (2002) as described by Kapravelou et al. (2015) using in the determination a gallic acid standard line with concentrations between 0 and 500 µg/mL.

Finally, the reductive capacity of Fe³⁺ to Fe²⁺ by the different extracts was determined spectrophotometrically by the technique of Duh et al. (1999) as described by Kapravelou et al. (2015) using in the determination a standard line of gallic acid with concentrations between 0 and 500 µg/mL.

TABLE 3

| Euphorbia lathyris | Yield (mg/g flour) | Total Polyphenols (µg gallic ac.equivalent /mg extract) | Reductive capacity (µg gallic ac.equivalent /mg extract) |
|---|---|---|---|
| Ripe seed flour without defatting | 42.9 ± 1.47 | 15.85 ± 0.21 | 9.71 ± 0.28 |
| Defatted ripe seed flour | 117.52 ± 9.22 | 33.52 ± 5.79 | 22.95 ± 1.92 |

As can be seen in Table 3, the yield obtained in relation to both extracts showed a large difference, the yield being significantly higher and more homogeneous with the defatted mature seed flour (117.52 ± 9.22 mg/g flour) versus the yield obtained from the undefatted mature seed flour (42.9 ±1.47 mg/g flour).

Antioxidant capacity was analyzed including biochemical studies of total polyphenols and reducing capacity. In terms of total polyphenols, extracts from undefatted mature seed flour showed values of 15.85 ± 0.21 µg gallic acid equivalents/mg extract, while extracts from defatted mature seed flour showed higher values (33.52 ± 5.79 µg gallic acid equivalents/mg extract), confirming the purity of working without lipid compounds which, in addition, in their elimination process, do not alter or eliminate phenolic compounds. Reducing capacity tests for the biochemical determination of antioxidant capacity showed values of 9.71 ± 0.28 µg gallic acid equivalents/mg extract in the case of flour from mature, undefatted seeds, and 22.95 ± 1.92 µg gallic acid equivalents/mg extract for defatted mature seed flour (see Table 3). Therefore, the antioxidant capacity of the extracts prepared with defatted mature seed flour was higher on the basis that polyphenols are obtained in higher amount.

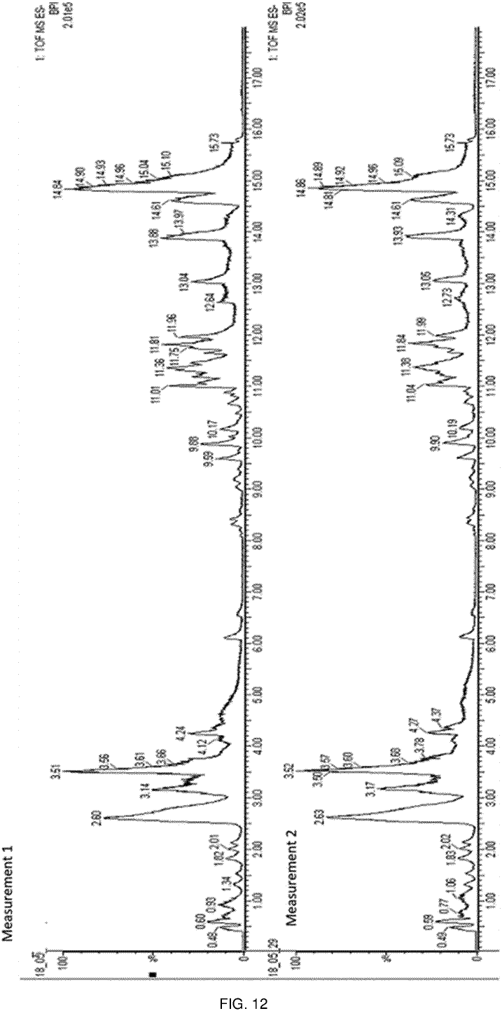

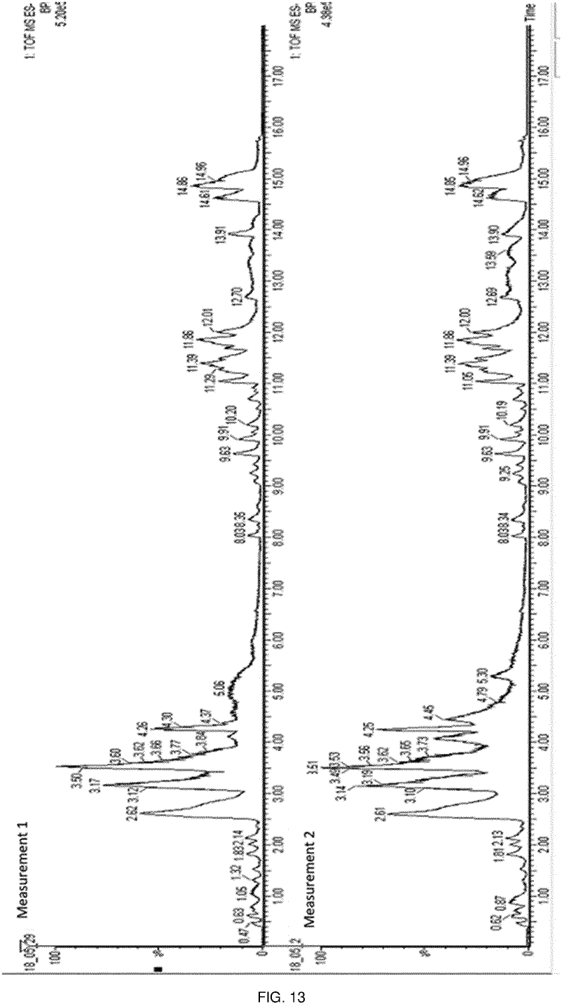

Chromatographic studies were carried out to determine the compounds present in the ethanolic extract of mature Euphorbia lathyris seed flours. The technique used consisted of an Ultra Performance Liquid Chromatography (UPLC) (ACQUITYH CLASSWATERS) coupled to a QTOF mass spectrometer (SYNAP G2. WATERS). FIGS. 12 and 13 show the graphs of the chromatographic replicates of the ethanolic extracts obtained either from the mature seed flour without defatting (FIG. 12) or defatted (FIG. 13). Tables 4 and 5 below show the main bioactive compounds identified in each of the extracts and the chromatographic data for each compound.

TABLE 4

Bioactive compounds from the ethanolic extract of undefatted mature seed flour of Euphorbia lathyris

| Compound | MF | [M-H]- | TR | PPM | %Reliab. |
|---|---|---|---|---|---|
| | | 177.0185 | 2.63 | -1.7 | |
| Esculetin | C₉H₆O₄ | | | | 90-100 |
| | | 177.0188 | 2.60 | 0.0 | |
| Euphorbetin | | 353.0293 | 3.14 | -1.1 | |
| | C₁₈H₁₀O₈ | | | | 90-100 |
| | | 353.0295 | 3.17 | -0.6 | |
| | | 445.1344 | 1.83 | -0.4 | |
| Gaultherin | C₁₉H₂₆O₁₂ | | | | 90-100 |
| | | 445.1353 | 1.82 | 1.6 | |
| | | 329.1744 | 6.11 | -2.7 | |
| Carnosol | C₂₀H₂₆O₄ | | | | 90-100 |
| | | 329.1758 | 6.13 | 1.5 | |
| | | 593.1512 | 3.81 | 1.0 | |
| Kaempferol-3- rutinoside | C₂₇H₃₀O₁₅ | | | | 90-100 |
| | | 593.1495 | 3.82 | -1.9 | |

TR: retention time; MF: molecular formula; PPM: error; MS: mass; %Reliab: reliability percentage

TABLE 5

Bioactive compounds of the ethanolic extract of defatted ripe seed flour of Euphorbia lathyris

| Compound | MF | [M-H]- | TR | PPM | % Reliab. |
|---|---|---|---|---|---|
| Esculetin | C₉H₆O₄ | 177.0181 | 2.62 | -4.0 | 90-100 |
| | | 177.0185 | 2.61 | -1.7 | |
| Euphorbetin | C₁₈H₁₀O₈ | 353.0298 | 3.17 | -0.6 | 90-100 |
| | | 353.0301 | 3.14 | 1.1 | |
| Gaultherin | C₁₉H₂₆O₁₂ | 445.1349 | 1.89 | 0.7 | 90-100 |
| | | 445.1351 | 1.81 | 1.1 | |
| Carnosol | C₂₀H₂₆O₄ | 329.1744 | 6.10 | -2.7 | 90-100 |
| | | 329.1750 | 6.11 | -0.9 | |
| Kaempferol-3-rutinoside | C₂₇H₃₀O₁₅ | 593.1510 | 3.86 | 0.7 | 90-100 |
| | | 593.1520 | 3.87 | 2.4 | |

TR: retention time; MF: molecular formula; PPM: error; MS: mass; %Reliab: reliability percentage.

Among the compounds present in the extracts studied, both from the mature, undefatted and defatted seed flour, esculetin, euphorbetin, gaultherin, nicotiflorin (Kaempferol-3-rutoside) and carnosol stand out. As previously discussed in the section, these are polyphenols that have been previously related to tumor activity in assays performed with the compounds independently, without having proven their activity as part of Euphorbia lathyris extracts. These polyphenols, independently obtained from commercial houses, have been related in previous studies with antitumor activity, without having been performed so far studies with extracts of Euphorbia lathyris, which are included among the assays presented below.

Likewise, the presence of these compounds has been corroborated and quantified by means of specific standards for each one: esculetin (Sigma-Aldrich, 68923) gaultherin (Cymitquimica,490-67-5), nicotiflorin (Cymitquimica,17650-84-9) and carnosol (Cymitquimica ,5957-80-2), highlighting the lack of the standard for euphorbetin, which being one of the predominant compounds in the chromatogram, is not currently marketed as an isolated compound. The data obtained are shown below in Tables 6 and 7.

TABLE 6

Quantification (ppb: µg/L) of bioactive compounds of replicate ethanolic extracts of mature, undefatted and defatted Euphorbia lathyris seed flour from known standards.

| | | Ppb (µg/L) | |
|---|---|---|---|
| | | Esculetin | Kaempferol-3-Rutinoside |
| Undefatted ripe seed flour of E. lathyris | Replica 1 | 985.9 | 59.8 |
| | Replica 2 | 1161.3 | 59.8 |
| | Replica 3 | 1197.2 | 61.1 |
| Defatted ripe seed flour of E. lathyris | Replica 1 | 2041.5 | 188.3 |
| | Replica 2 | 2131.8 | 327 |
| | Replica 3 | 2325.8 | 354 |

TABLE 7

Quantification (mg of bioactive compounds per 100 mg of extract) of the bioactive compounds of the replicates of ethanolic extracts of mature, undefatted and defatted seed flour of Euphorbia lathyris from known standards.

| | Concentration mg/ml | Esculetin (mg esculetin per each 100 mg extract) | Kaempferol-3-Rutinoside (mg kaempferol per each 100 mg extract) |
|---|---|---|---|
| Undefatted ripe seed flour of E. lathyris | 29.99 ± 4.46 | 0.4 ± 0.05 | 0.02 ± 0.003 |
| Defatted ripe seed flour of E. lathyris | 99.59 ± 13.84 | 0.21 ± 0.023 | 0.03 ± 0.006 |

Of these patterns, two appeared in majority, esculetin and kaempferol-3-ruthinoside (see Table 6). As in the yield, the defatted mature seed flour contained more of these compounds, with 2166.37 ppb (µg/L) of esculetin compared to 1114.8 ppb (µg/L) of the same in the extract of the undefatted mature seed flour. The same is true for kamepferol, 289.78 ppb (µg/L) in the defatted mature seed flour extract versus 60.23 ppb (µg/L) in the undefatted mature seed flour extract (Tables 6 and 7).

### Example 6. Determination of the Antitumor Capacity of the Extracts

To determine the antitumor capacity of the extracts, the cell lines T84 (human colon adenocarcinoma cell line) and HCT15 (human colon adenocarcinoma cell line resistant to chemotherapy) were cultured. As a control, the CCD18 cell line (non-tumorigenic human colon epithelial cell line) was selected.

The ethanolic extracts were previously evaporated to avoid the toxicity caused by ethanol on the cell lines.

In addition, once evaporated, a part was lyophilized to know the amount of extract obtained and to quantify its concentration (mg/ml) with which the different concentrations to be tested will be calculated. The cell cultures were exposed to increasing concentrations of the evaporated ethanolic extract of mature, undefatted and defatted seed flour, which made it possible to determine the inhibitory dose 50 (IC50) (concentration of the extract at which it inhibits 50% of the cell population). The results obtained are shown in Table 8.

TABLE 8

Antitumor capacity of extracts from mature, undefatted and defatted Euphorbia lathyris seed flour in vitro at 72 hours in different colon cancer lines.

| Euphorbia lathyris | IC₅₀ (µg/mL) | | |
|---|---|---|---|
| | T84 | HCT15 | CCD18 |
| Ethanolic extract of mature seed flour without defatting | 11.04 ± 1.63 | 34.26 ± 1.1 | 388.36 ± 30.14 |
| Ethanolic extract of defatted ripe seed flour | 16.29 ± 2.54 | 72.9 ± 1.27 | 266.02 ± 18.5 |

T84: human colon adenocarcinoma cell line; HCT15: human colon adenocarcinoma cell line resistant to chemotherapy; CCD18: human healthy colon cell line. IC50: concentration that inhibits 50% of the cells.

As can be seen in the table above, for the mature seed extract, the IC50s were as follows: 11.04 ± 1.63 µg/ml in T84, 34.26 ± 1.1 µg/ml in HCT15. In the case of the normal colon line CCD18, the IC50 was much higher (388.36 ± 30.14 µg/ml,) indicating lower activity and thus toxicity in this cell type compared to tumor cells.

For extracts from defatted mature seed flour, the IC50s were: 16.29 ± 2.54 µg/ml in T84, 72.9 ± 1.27 µg/ml in HCT15 and 266.02 ± 18.5 µg/ml in CCD18.

In view of the results, we observed that both the extract of the mature seed flour, undefatted and defatted, have a high antiproliferative activity with very low IC50s, regardless of whether the mature seed flour is undefatted or defatted, so that the defatting process does not affect the antitumor capacity of the extract obtained. It is noteworthy in both cases, the difference between the IC50 of the tumor lines (T84 and HCT15) and the non-tumor line (CCD18), the latter being much higher than the tumor lines.

Because both the undefatted and defatted mature seed flour extracts possess similar antitumor activity, but the defatted mature seed flour extracts have higher yields and higher concentrations of polyphenolic compounds, we used the ethanolic extract of defatted mature seed flour to perform the remaining molecular tests detailed below.

Finally, and based on the previous results, the ethanolic extract of defatted mature seed flour, selected for the rest of the molecular tests, was tested in glioblastoma multiforme and pancreatic adenocarcinoma cell lines, two of the most aggressive types of cancer, with the worst prognosis and for which there are few therapeutic possibilities. For this purpose, cell lines A-172 (human glioblastoma cell line), SF-268 and SK-N-SH (human glioblastoma cell lines resistant to chemotherapy), as well as the Panc-1 cell line (human pancreatic adenocarcinoma cell line) were cultured. Using the same procedure described above, the IC50s of the cell lines under study were determined. The results are shown in Table 9.

TABLE 9

Results of the use of ethanolic extract of defatted mature seed flour tested on glioblastoma multiforme and pancreatic adenocarcinoma cell lines.

| Euphorbia lathyris | IC₅₀ (ug/ml) | | | |
|---|---|---|---|---|
| | SF-268 | SK-N-SH | A-172 | Panc-1 |
| Ethanolic extract of defatted ripe seed flour | 39.33 ± 13.2 | 71.42 ± 13.6 | 18.58 ± 1.64 | 185.76 ± 25.8 |

A-172 (human glioblastoma cell line), SF-268 and SK-N-SH (human chemotherapy-resistant glioblastoma cell lines), as well as the Panc-1 cell line (human pancreatic adenocarcinoma cell line).

Thus, the IC50 in human glioblastoma cell lines were: 39.33 ± 13.2 µg/ml in SF-268, 71.42 ± 13.6 µg/ml in SK-N-SH and 18.58 ± 1.64 µg/ml in A-172 while in the pancreatic adenocarcinoma derived line Panc-1, the IC50 was 185.76 ± 25.8 µg/ml. These results led to the conclusion that the ethanolic extract of defatted mature seed flour, in addition to exhibiting high antitumor activity against colon cancer, also possesses high antitumor activity against glioblastoma and pancreatic adenocarcinoma cell lines.

### Example 7- Molecular Study of Proteins Related to Cell Death

To elucidate the mechanisms by which the extracts of the present invention act, the cell death pathway (apoptosis) mediated by caspases, mainly caspase 8 (extrinsic pathway), caspase 9 (intrinsic pathway) and caspase 3, was studied by western blot, using β-actin as an endogenous control.

For this purpose, colon tumor line cells (T84) were cultured with an IC50 of the ethanolic extract obtained from defatted mature seed flour and after 72 hours, the cells were harvested for protein extraction.

To perform the western blot assay, 40 µg of protein from cells treated with the ethanolic extract, as well as from control cells, were loaded onto an SDS-PAGE electrophoresis polyacrylamide gel in a Mini Protean II cell (Bio-Rad, Hercules, CA). Once the proteins were separated by electrophoresis, they were transferred to a nitrocellulose membrane that was supplied with 20 V at room temperature for 1 h. These membranes were treated with blocking solution (PBS-Tween + 5% milk powder) for 1 h and then, after 2 washes with PBS-Tween, incubated with the primary antibody [rabbit polyclonal IgG anti-caspase-3 (1:500 dilution), anti-caspase-8 (1:1000 dilution) and anti-caspase-9 (1:1000 dilution); Santa Cruz Biotechnology, Santa Cruz, CA). Incubate overnight at 4° C. After incubation time, two washes are performed and incubated for 1 h at room temperature with peroxidase-conjugated secondary antibody. Finally, the proteins are detected by ECL (enhanced chemiluminescence) (Bonnus, Amersham, Little Chalfont, UK) (Ortíz et al. 2009).

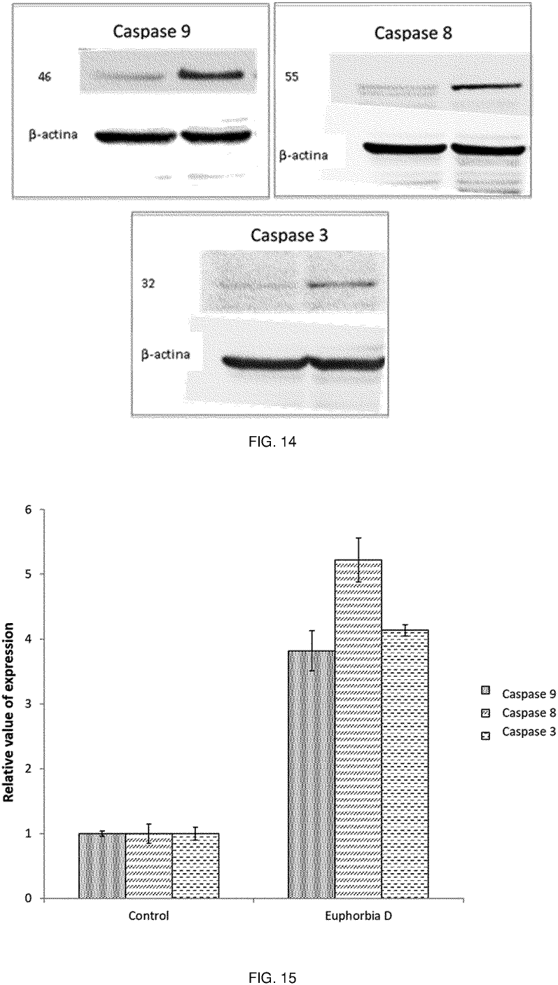

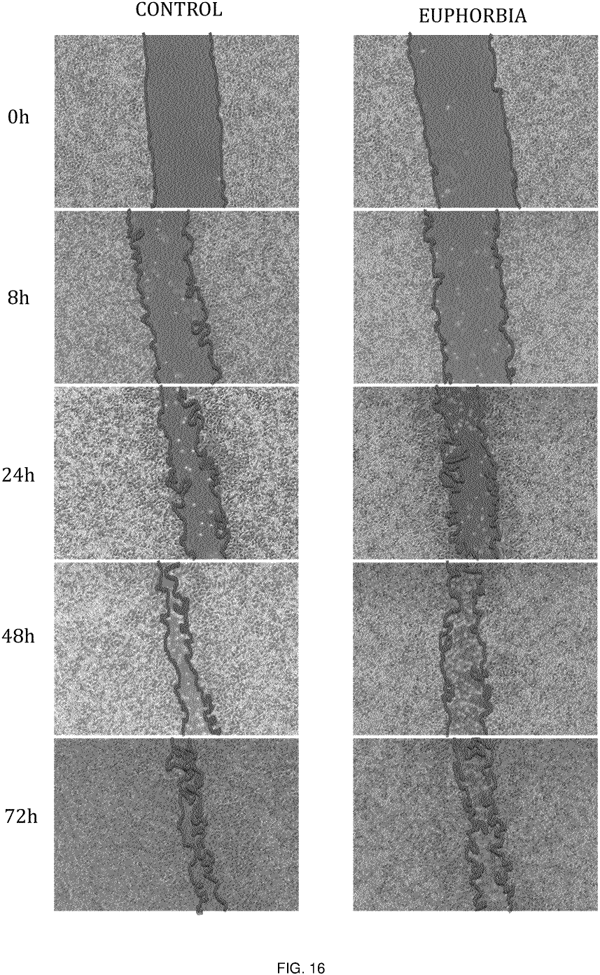

Once the Western Blot was performed (see FIG. 14), the bands obtained in the gels were analyzed using Quantity One Bio-Rad analytical software, confirming that the ethanolic extract of defatted mature seed flour of Euphorbia lathyris produces cell apoptosis mediated by the caspases pathway, being expressed up to four times more than the control (FIG. 15).

Therefore, we can conclude that the ethanolic extract from defatted seeds produces cell death by apoptosis, both intrinsically and extrinsically.

### Example 8 Determination of the Migration Capacity of Tumor Cells

To determine the migration capacity of tumor cells, and therefore their invasiveness and ability to generate metastasis, an in vitro migration assay was performed. For this purpose, cells of the T84 colon tumor line were seeded in twelve-well plates at 100% confluence. At 24 hours, the gap or “wound” is made manually with a sterile tip. This gap consists of generating, in the well, a cell-free space (gap) in the middle of a cell monolayer and being able to observe and quantify the cell displacement on it (Grada et al, 2017). Once carried out and verified that the gap is free of cells, it is changed to medium without fetal bovine serum so that the cells around the gap stop multiplying and a non-cytotoxic dose of the ethanolic extract from the defatted mature seed flour is added in triplicate. Pictures are taken at different times (0, 8, 24, 24, 48 and 72 hours) to observe cell migration, compared to the control (untreated cells). Images of the results obtained can be seen in FIG. 16.

To evaluate the effect of the ethanolic extract, the percentage of migration is calculated by measuring the area of the gap still free of tumor cells at the different times at which the images were taken using the Image J software.

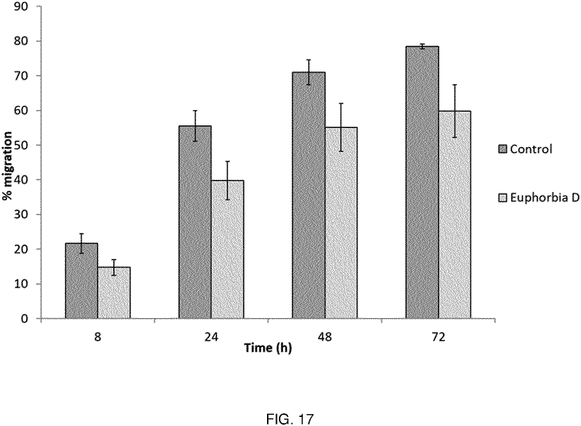

After performing the assay, it can be concluded that, at non-cytotoxic doses of the extract, colon tumor cells migrate significantly less than controls, and therefore, the extract of the present invention, at non-cytotoxic doses, would significantly slow down invasiveness and metastasis formation. In fact, already at 8 hours the slowing down of the migration of tumor cells exposed to non-cytotoxic doses of the extract of the present invention was significantly evident. At 72 hours, with the completion of the experiment, this decrease was greater, decreasing from 79% migration in controls to 60% with the extract, decreasing by about 20% (FIG. 17).

### BIBLIOGRAPHIC REFERENCES

Aliebrahimi S, Kouhsari S, Arab S, Shadboorestan A, Ostad S. Phytochemicals, withaferin A and carnosol, overcome pancreatic cancer stem cells as c-Met inhibitors. Biomedicine & Pharmacotherapy. 2018; 106:1527-1536.

Amin A, Kucuk O, Khuri F, Shin D. Perspectives for Cancer Prevention With Natural Compounds. Journal of Clinical Oncology. 2009;27(16):2712-2725.

Arora et al., Molecular Cancer, 2016, 15(1),1-15

Berman B. New developments in the treatment of actinic keratosis: focus on ingenolmebutate gel. Clinical, Cosmetic and Investigational Dermatology. 2012;111.

Berrino F, De Angelis R, Sant M, Rosso S, Lasota M, Coebergh J et al. Survival for eight major cancers and all cancers combined for European adults diagnosed in 1995-99: results of the EUROCARE-4 study. The Lancet Oncology. 2007;8(9):773-783.

Bicchi C, Appendino G, Cordero C, Rubiolo P, Ortelli D, Veuthey JL. HPLC-UV and HPLC-positive-ESI-MS analysis of the diterpenoid fraction from caper spurge (Euphorbia lathyris) seed oil. Phytochem Anal. 2001; 12(4):255-62.

Cabeza et al., AAPS PharmSciTech 2020, 21(5), 1-15).

Cruz L, de Oliveira T, Kanunfre C, Paludo K, Minozzo B, Prestes A et al. Pharmacokinetics and cytotoxic study of euphol from Euphorbia umbellata (Bruyns) Pax latex. 2018.

Dewanto V, Wu X, Adom KK and Liu RH, Thermal processing enhances the nutritional value of tomatoes by increasing total antioxidant activity. J Agric Food Chem 50:3010-3014 (2002).

Duan FP. Wang YZ, Li CX. Chemical composition and biological activity analysis of semen euphorbiae petroleum ether extracts. J. Chem. Pharm. Res. 2014; 6, 745-749.

Duh PD, Tu YY and Yen GC, Antioxidant activity of the aqueous extract of harn jyur (Chrysanthemum morifolium Ramat). Lebensm-Wiss Technol 32:269-277 (1999).

Evans F, Taylor S. Pro-inflammatory, tumor-promoting and anti-tumor diterpenes of the plant families euphorbiaceae and thymelaeaceae. Chemischerlnformationsdienst. 1984;15(11).

Fan L, Zhu H, Tao W, Liu L, Shan X, Zhao M, Sun D. Euphorbia factor L2 inhibits TGF-β-induced cell growth and migration of hepatocellular carcinoma through AKT/STAT3. Phytomedicine, 2019, 62:152931.

Franceschi S, Wild C. Meeting the global demands of epidemiologic transition - The indispensable role of cancer prevention. Molecular Oncology. 2012;7(1):1-13.

Goyal S, Gupta N, Chatterjee S, Nimesh, S. Natural Plant Extracts as Potential Therapeutic Agents for the Treatment of Cancer. Current Topics in Medicinal Chemistry. 2017; 17(2):96-106.

Grada A, Otero-Vinas M, Prieto-Castrillo F, Obagi Z, Falanga V. Research Techniques Made Simple: Analysis of Collective Cell Migration Using the Wound Healing Assay. J Invest Dermatol. 2017;137(2): e11-e16. doi: 10.1016/j.jid.2016.11.020

Hargreaves BJ. Succulents of Malawi. Society of Malawi Journal 1968;31: 31-44.

HefnyGad M, Tuenter E, El-Sawi N, Younes S, El-Ghadban E, Demeyer K et al. Identification of some Bioactive Metabolites in a Fractionated Methanol Extract from Ipomoea aquatica (Aerial Parts) through TLC, HPLC, UPLC-ESI-QTOF-MS and LC-SPE-NMR Fingerprints Analyses. PhytochemicalAnalysis. 2018;29(1):5-15.

Huang W, Cai Y, Zhang Y. Natural Phenolic Compounds From Medicinal Herbs and Dietary Plants: Potential Use for Cancer Prevention. Nutrition and Cancer. 2013;62(1):1-20.

Jiao W y col., “Studies on chemical constituents in seeds of Euphorbia lathyris”, Chinese Traditional and Herbal Drugs, 2010, vol. 51, paginas 181-187

Kapravelou G, Martínez R, Andrade AM, López Chaves C, López-Jurado M, Aranda P, Arrebola F, Cañizares FJ, Galisteo M, Porres JM. Improvement of the antioxidant and hypolipidaemic effects of cowpea flours (Vigna unguiculata) by fermentation: results of in vitro and in vivo experiments. J Sci Food Agric 95(6):1207-1216 (2015).

Kupchan S, Uchida I, Branfman A, Dailey R, Fei B. Antileukemic principles isolated from euphorbiaceae plants. Science. 1976;191(4227):571-572.

Labianca R, Merelli B. Screening and Diagnosis for Colorectal Cancer: Present and Future. Tumori Journal. 2010;96(6):889-901.

Latansio de Oliveira et al., Journal of Ethnopharmacology, 265 (August 2020

Lee R, Jeon Y, Cho J, Jang J, Kong I, Kim S et al. Esculetin exerts anti-proliferative effects against non-small-cell lung carcinoma by suppressing specificity protein 1 in vitro. General physiology and biophysics. 2017;36(01):31-39.

Levey M. Medieval Arabic Toxicology: The Book on Poisons of ibn Wahshiya and Its Relation to Early Indian and Greek Texts. Transactions of the American Philosophical Society. 1966;56(7):1.

Liao S, Zhan Z, Yang S, Yue J. Lathyranoic Acid A: First Secolathyrane Diterpenoid in Nature from Euphorbia lathyris. 2005.

Loree J, Kopetz S. Recent developments in the treatment of metastatic colorectal cancer. Therapeutic Advances in Medical Oncology. 2017;9(8):551-564.

Martínez-Ballesta et al., Foods, 2018, 7(5), 1-29

Martins et al., Journal of Ethnopharmacology, 255, February 2020

Meng X, Zhao X, Long Z, Yuan Y, Zhuang H, Bi K, Chen X. A sensitive liquid chromatography-mass spectrometry method for simultaneous determination of three diterpenoid esters from Euphorbia lathyris L. in rat plasma. J Pharm Biomed Anal. 2013; 72:299-305.

Nam, AK, Lee, SK. Evaluation of cytotoxic potential of natural products in culture human cancer cells. Nat. Prod. Sci. 2000; 6, 183-188

Nambudiri N, Nambudiri V. Euphorbia peplus:18th-Century Insights on a 21st-Century Therapy. JAMA Dermatology. 2013;149(9):1081.

Ortiz R, Prados J, Melguizo C, et al. The cytotoxic activity of the phage E protein suppress the growth of murine B16 melanomas in vitro and in vivo. J Mol Med (Berl). 2009;87(9):899-911. doi:10.1007/s00109-009-0493-9

Rivas-Morales C, Oranday-Cardenas MA, Verde-Star MJ. Investigación de plantas de importancia medica. Ed. IV. Universidad autonoma de nuevo León, Mexico. 2016; ISBN: 978-84-94673-7-0.

Rodríguez-Ruiz et al., Langmuir, 2013, 29(26), 8213-8221.

Sdayria J, Rjeibi I, Feriani A, Ncib S, Bouguerra W, Hfaiedh N et al. Chemical Composition and Antioxidant, Analgesic, and Anti-Inflammatory Effects of Methanolic Extract of Euphorbia retusa in Mice. Pain Research and Management. 2018; 2018:1-11.

Shi Q, Su X, Kiyota H. Chemical and pharmacological research of the plants in genus Euphorbia. ChemicalReviews. 2008; 108:4295-4327.

Silva V, Rosa M, Tansini A, Oliveira R, Martinho O, Lima J et al. Invitro screening of cytotoxic activity of euphol from Euphorbiatirucalli on a large panel of human cancer-derived cell lines. 2018.

Stannard, J. A fifteenth-century botanical glossary. Isis 1964;55: 353-367.

Teng Y, Wang Y, Hsu P, Xin G, Zhang Y, Morris-Natschke S et al. Mechanism of action of cytotoxic compounds from the seeds of Euphorbia lathyris. Phytomedicine. 2018; 41 :62-66.

Thapliyal A, Krishen R, Chandra A. Overview of Cancer and Medicinal Herbs used for Cancer Therapy. Asian Journal of Pharmaceutics. 2018;12(1).

Turkekul K, Colpan R, Baykul T, Ozdemir M, Erdogan S. Esculetin Inhibits the Survival of Human Prostate Cancer Cells by Inducing Apoptosis and Arresting the Cell Cycle. Journal of Cancer Prevention. 2018;23(1):10-17.

Vasas A, Hohmann J. Euphorbia Diterpenes: Isolation, Structure, Biological Activity, and Synthesis (2008-2012). Chemical Reviews. 2014;114(17):8579-8612.

Wang CJ, Hsieh YJ, Chu CY, Lin YL, Tseng TH. Inhibition of cell cycle progression in human leukemia HL-60 cells by esculetin. Cancer Lett. 2002; 183:163-8.

Wang Q, Zhen YQ, Gao F, Huang S, Zhou XL. Five new diterpenoids from the seed of Euphorbia lathyris. Chem Biodivers. 2018.

Wang et al., BioMed Research International, 2020

Wong F, Chai T, Xiao J. The influences of thermal processing on phytochemicals and possible routes to the discovery of new phytochemical conjugates. Critical Reviews in Food Science and Nutrition. 2018:1-6.

Zhang L, Wang C, Meng Q, Tian Q, Niu Y, Niu W. Phytochemicals of Euphorbia lathyris L. and Their Antioxidant Activities. Molecules. 2017; 22(8). pii: E1335.

Zhou M. et al., Journal of Asian Natural Products Research, 2020, 0(0), 1-7

Zhu A, Zhang T, Wang Q. The phytochemistry, pharmacokinetics, pharmacology and toxicity of Euphorbia semen. 2018.

1. A composition characterized in that it comprises:
a) 0.2 to 30 mg of esculetin per g of total composition and
b) 2 to 15 mg of euphorbetin per g of total composition.

2. The composition of claim 1, characterized in that it comprises from 2 to 25 mg of esculetin per g of total composition, preferably from 10 to 20 mg of esculetin per g of total composition.

3. The composition of claim 1, characterized in that it comprises from 5 to 10 mg of euphorbetin per g of total composition.

4. The composition of claim 1, wherein said composition is in the form of an ethanolic extract of a plant from genus Euphorbia.

5. The composition of claim 4, wherein the plant from genus Euphorbia is selected from Euphorbia lathyris, Euphorbia anguiata, Euphorbia cyparissias, Euphorbia dulcis, Euphorbia helioscopia, Euphorbia maculata, Euphorbia peplus, Euphorbia prostrata, Euphorbia valliniana or Euphorbia verrucosa , preferably from Euphorbia lathyris.

6. The composition of claim 4, which further comprises gaultherin, kaempferol-3-rutinoside and carnosol.

7. A drug delivery system comprising a plurality of calcium phosphate nanoparticles (CP-NPs) surface functionalized with the composition of claim 1-6, characterized in that said CP-NPs comprise esculetin and euphorbetin adsorbed to the surface of the nanoparticle.

8. The drug delivery system of claim 7, characterized in that it said CP-NPs comprise:
a) 0.1 to 7 mg of esculetin per g of the functionalized CP—NPs, preferably 0.2 to 5 mg of esculetin per g of the functionalized CP—NPs and
b) 2 to 10 mg of euphorbetin per g of the functionalized CP—NPs, preferably 3 to 8 mg of euphorbetin per g of the functionalized CP—NPs.

9. The drug delivery system of claim 7, characterized in that the average size of the CP-NPs is between 20 and 50 nm, preferably between 30 to 40 nm.

10. The composition of claim 1 or the drug delivery system of claim 7 te-9-for use as a medicament.

11. The composition of claim 1 or the drug delivery system of claim 7 for use in the treatment of cancer, preferably colorectal, pancreatic or glioblastoma.

12. Pharmaceutical composition comprising the composition of claim 1 or the drug delivery system of claim 7 and at least a pharmaceutically acceptable excipient or carrier, and optionally a further active compound.

13. A process for obtaining an ethanolic plant extract from the flour of mature seeds of Euphorbia according to claim 4, comprising the steps of:
a) grinding the seed to obtain flour;
b) extracting the flour from step (a) by means of a cold hydroalcoholic extraction solution at acid pH,
c) optionally, defatting the mature seed by mechanical cold pressing prior to steps (a) and (b).

14. The process according to claim 13, wherein:
a) the seed is ground to flour with a particle size between 100 pm and 150 gm; and
b) the flour obtained in step a) is extracted with the extraction solution under the following operating conditions:
i. temperature equal to 4° C.,
ii. under nitrogen atmosphere,
iii. the extraction solution is composed of ethanol, double distilled water and hydrochloric acid in the proportions 50:50:0.2 by volume,
iv. pH equal to 2, and/or
v. the mixture of the flour and the extraction solution is kept stirred for 30 minutes after conditions i to iv. have been reached,
and wherein the ethanolic extract is obtained by centrifuging the mixture of extraction solution and flour and collecting the supernatant.

15. The process according to claim 13 , wherein the degreasing step of the mature seed is carried out at a temperature of 40 to 50° C. and an extraction rate of 2 to 3 kg of seed/hour.

16. The process according to claim 14 , wherein:
vi. the precipitate resulting from centrifuging the mixture of flour and extraction solution is resuspended in extraction solution,
vii. the suspension obtained is again stirred for 30 minutes under the conditions of step (b) as defined in claim 14,
viii. the suspension is centrifuged and the supernatant is collected; and
ix. the ethanolic extract results from mixing the supernatant obtained in (iii) with the first supernatant obtained.

17. The process according to claim 13, comprising a final additional step wherein the ethanol is partially or completely evaporated from the ethanolic extract obtained.
